(19) United States
(12) Patent Application Publication
(10) Pub. No.: US 2025/0295773 A1
(43) Pub. Date: Sep. 25, 2025

(54) COMPOSITIONS COMPRISING IL-15, IL-15 RECEPTOR ALPHA AND THE INTRACELLULAR SIGNALING DOMAIN OF CD2 FOR IMMUNE CELL THERAPY

(71) Applicant: MILTENYI BIOTEC B.V. & CO. KG, Bergisch Gladbach (DE)

(72) Inventors: Congcong Zhang, Bergisch Gladbach (DE); Jonathan Druge, Bergisch Gladbach (DE); Nina Moker, Bergisch Gladbach (DE); Melissa Quadflieg, Bergisch Gladbach (DE); Mario Assenmacher, Bergisch Gladbach (DE)

(73) Assignee: MILTENYI BIOTEC B.V. & CO. KG, Bergisch Gladbach (DE)

(21) Appl. No.: 18/864,451

(22) PCT Filed: May 9, 2023
(86) PCT No.: PCT/EP2023/062306

§ 371 Date: Nov. 8, 2024

(30) Foreign Application Priority Data
May 10, 2022 (EP) 22172578.1

(51) Int. Cl. A61K 40/31; A61K 40/42; A61P 35/00; C07K 14/54; C07K 14/725

(52) U.S. Cl. CPC A61K 40/31; A61K 40/4217; A61P 35/00; C07K 14/5443; C07K 14/7051; A61K 2239/48; C07K 2319/03

(57) ABSTRACT

The present invention provides a composition comprising A) a nucleic acid sequence comprising encoding I) a) a fusion protein comprising from N-terminus to C-terminus i) IL-15Rα and, ii) the intracellular signaling domain of CD2, and b) IL-15, or II) a fusion protein comprising from N-terminus to C-terminus i) IL-15, ii) a linker, iii) IL-15Ra, and iv) the intracellular signaling domain of CD2, or B) a first nucleic acid sequence and a second nucleic acid sequence, said first nucleic acid sequence comprising encoding a fusion protein comprising from N-terminus to C-terminus i) IL-15Ra and ii) the intracellular signaling domain of CD2, said second nucleic acid sequence comprising encoding IL-15. Said composition may additionally comprise a transgene such as a CAR. Also disclosed are immune cells expressing the nucleic acids of said composition.

### FIELD OF THE INVENTION

The invention relates to immune cells for use in immunotherapy, in particular to immune cells that include a CD2 signaling moiety into the IL-15:IL-15Rα complex.

### BACKGROUND OF THE INVENTION

Although clinical success has been achieved by using T cell- or NK cell-based therapies against cancer or viral infectious disease, several major challenges limit their full therapeutic potentials. The inefficient persistence and expansion of infused immune cells in patients are negatively correlated with the duration of clinical responses. Immunosuppressive tumor microenvironment (TME) strongly induce dysfunction of immune effector cells, resulting in poor proliferation and cytolytic activity.

The use of chimeric antigen receptor (CAR)-expressing immune cells re-directed to specifically recognize and eliminate malignant cells, greatly increased the scope and potential of adoptive immunotherapy and is being assessed for new standard of care in certain human malignancies. CARs are recombinant receptors that typically target surface molecules in a human leukocyte antigen (HLA)-independent manner. Generally, CARs comprise an extracellular antigen recognition moiety, often a single-chain variable fragment (scFv) derived from antibodies, a Fab fragment or a nanobody, linked to an extracellular spacer, a transmembrane domain and intracellular co-stimulatory and signaling domains.

Interleukin (IL)-15 belongs to the type I cytokine family and regulates the homeostasis of innate and adaptive immunity. IL-15 exhibits biological functions on many diverse immune cell types, including natural killer (NK) cells, αβT cells, γδT cells, invariant NKT (iNKT) cells, B cells, monocytes, macrophages, dendritic cells (DCs), neutrophiles, eosinophiles and mast cells. IL-15 plays an essential role in the development, survival, proliferation, activation of NK cells and T cells.

The heterotrimeric IL-15 receptor complex is consisting of IL-2 receptor beta chain (IL-2Rβ, also known as CD122), common cytokine receptor gamma chain (γc, also known as CD132) and a unique receptor subunit IL-15 receptor alpha chain (IL-15Rα, CD215). In the prevalent view, IL-15 is associated with IL-15Rα expressed by antigen-presenting cells, such as dendritic cells (DCs), with high affinity and presented in trans to IL-2RB and γc expressed on the surface of effector immune cells. IL-15 then exhibits its functions in effector immune cells via the heterodimer of IL-2Rβ and γc by triggering janus kinases (JAK) and signal transducer and activator of transcription (STAT) signaling molecules. However, the topology analysis reveals that IL-15 is capable of forming the signaling complex with IL-15Rα and IL-2Rβ/γc on a single cell and induce efficient signaling via cis-presentation. Indeed, IL-15Rα has been found be present in NK cells and can present IL-15 in cis to activate NK cells.

CD2 is a transmembrane glycoprotein of 327 amino acids and a molecular weight of 40 kDa in humans. This receptor is structured as an extracellular domain, a transmembrane domain and a cytoplasmic tail responsible for signal transduction. The extracellular domain of CD2 consists of 2 immunoglobulin (Ig)-like domains, with the distal one recognizing and binding its ligand: lymphocyte-associate antigen 3 (LFA3, also known as CD58). CD2 can also bind to CD48 and CD59 with low affinity. In addition, CD15 has been reported as a CD2 ligand.

Several types of immune cells express CD2, including T cells, NK cells, thymocytes and dendritic cells (DCs). As an adhesion molecule, CD2 is recruited and accumulated in the immune synapse (IS).

CD2 enhances NK cell lytic activity against tumor cells when activated by its ligands and increases inflammatory cytokine production. CD2 can stimulate NK cells by binding to the ligands expressed on neighboring NK cells.

CD2 serves as the primary costimulatory receptor for T cell activation, compared with other costimulatory receptors including tumor necrosis factor receptor superfamily member 9 (TNFRSF9; also known as 4-1BB, CD137), Natural killer group 2D (NKG2D; also known as CD314), inducible T-cell costimulatory (ICOS, also known as CD278), tumor necrosis factor receptor superfamily member 7 (TNFRSF7; also known as CD27). CD2 contributes to TCR signaling intensity in T cells by lowering the TCR activation threshold.

Liu et al. (2018, Leukemia 32, 520-531) used cord blood (CB)-derived NK cells to generate CD19-specific CAR NK cells coexpressing soluble human IL-15 and an inducible suicide gene caspase 9 (iC9). The resulted iC9/CAR.19/IL15-transduced CB NK cells showed in vitro and in vivo anti-tumor activity. In a subsequential phase I clinical trial (Liu et al 2020, N. Engl. J. Med. 382, 545-553), clinical response has been observed in 8 out of 11 patients treated with iC9/CAR.19/IL15-transduced CB NK cells, including complete remission in 7 patients. Long-term persistence of iC9/CAR.19/IL15-transduced CB NK cells have been demonstrated by quantitative polymerase chain reaction (qPCR) method for at least 12 months. However, the low levels of persistent iC9/CAR.19/IL15-transduced CB NK cells did not prevent relapse in patients. The in vitro assay showed only about 1.2-fold expansion of iC9/CAR.19/IL15-transduced CB NK cells within the first 3 days in the absence of exogenous cytokine support, and the NK cell number started to decline afterwards as shown in Liu et al., (2018, Leukemia 32, 520-531). The low proliferation rate of CAR NK cells expressing native IL-15 may limit their therapeutic effects.

US2016/0158285A1 disclosed a membrane-bound chimeric IL-15. The membrane-bound chimeric IL-15 consists of the human IL-15 cDNA sequence fused to the full-length IL-15Rα cDNA sequence via a 26 amino acid linker. A (FLAG)3 epitope tag is added at the C-terminal of the membrane-bound chimeric IL-15. Coexpressing the membrane-bound chimeric IL-15 in CD19-specific CAR T cells improved T-cell persistence even in the absence of CAR activation and rendered CAR-T cells a phenotypical and transcriptional profile close to T-memory stem cells (Tscm). There was no autonomous growth or transformation was observed in their study. The in vitro assay showed no expansion of CAR-T cells expressing the membrane-bound chimeric IL-15 after the withdrawal of antigen and exogenous cytokine support. However, the number of CAR-T cells expressing the membrane-bound chimeric IL-15 declined significantly slower than that of CAR-T cells without the membrane-bound chimeric IL-15 expression. Engineered induced pluripotent stem cell (iPSC)-derived NK cells have been engineered with a membrane-bound IL-15/IL-15 receptor fusion protein (IL-15RF), which fuses IL-15 to IL-15 receptor alpha via a flexible linker, to support the in vivo persistence and functions of iPSC-NK cells (Kaufman et al., 2018, Blood 132, 4541-4541; Woan et al., 2021; Cell Stem Cell 28, 2062-2075.e5). Such iPSC-NK cells have been applied in Phase I clinical trials (Bachanova et al., 2021, Lymphoma. Blood 138, 823-823).

U.S. Pat. No. 10,774,311B2 discloses another membrane-bound IL-15 (mbIL15) by fusing human IL-15 to CD8α hinge and transmembrane domain. The whole construct is transferred to cell membrane by a CD8α signal peptide. NK cells expressing mbIL15 displayed strong in vitro and in vivo anti-tumor activity (Imamura et al., 2014, Blood 124, 1081-1088). Autonomous mbIL15 expression improved NK cell persistence by maintaining the engineered NK cells at detectable level 75 days after initiation of the culture in the absence of exogenous cytokine supply. However, median cell expansion of 2.05-fold on day 21 was achieved for mbIL15-expressing NK cells even in the presence of low dose of IL-2 (10 IU/mL). Under the same condition, 80% of mock transduced NK cells survived on day 21.

WO2019/160956A1 discloses a single nucleic acid molecule consisting of a CAR, an IL-15 receptor (IL-15R) and IL-15, which are linked by linkers encoding self-cleavage sites such as P2A or T2A. This nucleic acid molecule is supposed to use to generate immune cells coexpressing all three cassettes in the same cell. CAR-T cells coexpressing IL-15/IL-15R exhibited a naïve phenotype with CCR7+ and CD45RO− 60 days in culture without antigen stimulation. Upon antigen restimulation, CAR-T cells coexpressing IL-15/IL-15R exhibited a possessed less differentiate phenotype (CCR7+/−, CD45RO+), compared with CAR-T cells alone. CAR-T cells coexpressing IL-15/IL-15R showed superior proliferation upon antigen stimulation compared with CAR-T cells alone and controlled tumor growth in AsPC1 pancreatic cancer xenograft mouse models. However, CAR-T cell number did not expand without exogenous cytokine supply and antigen stimulation.

There is a need in the art for improved or alternative immune cells for immune therapy such as CAR immune cell therapy.

### BRIEF DESCRIPTION OF THE INVENTION

Surprisingly it was found that immune cell and/or CAR-engineered immune cell functionality can be augmented by including CD2 signaling moiety into the IL-15:IL-15Rα complex of the immune cell. Ectopic expression of the IL-15:IL-15Rα-CD2 constructs (herein also referred to as IL15/215.2 (a polypeptide comprising IL-15 and another polypeptide comprising IL-15Rα (CD215) and the intracellular signaling domain of CD2) and IL15.215.2 (a polypeptide comprising IL-15, IL15Rα and the intracellular signaling domain of CD2)) provided signaling to enhance survival, proliferation, anti-tumor activity of immune cells. Moreover, the presence of the IL-15:IL-15Rα-CD2 constructs can also expand said genetically modified immune cells in the ex vivo culture without exogenous cytokine support.

Therefore, the present invention provides compositions comprising nucleic acids sequences that encode IL-15, IL15Rα and CD2 in constructs as disclosed herein, and immune cells that comprise said nucleic acids.

Also disclosed is an in-vitro method for expanding said genetically engineered immune cell comprising the step of expanding said genetically engineered immune cell without exogenous cytokine support in a culture medium that comprises said genetically engineered immune cell.

### BRIEF DESCRIPTION OF THE DRAWINGS

FIG. 1: Schematic representations of the CAR construct (A), the complexes of IL-15:IL-15Rα variants (B) and lentiviral transfer plasmids encoding CARs and the complexes of IL-15:IL-15Rα variants under the transcriptional control of the human elongation factor 1 alpha (EF1α) promoter (C).

FIG. 2: Flow cytometric analysis of the expression of CAR (upper panels) and CD215 (lower panels) on CAR NK cells expressing the complexes of IL-15:IL-15Rα variants. Data from a representative experiment are shown.

FIG. 3: Production of soluble IL-15 and the heterodimers comprising IL-15 and soluble form of IL-15Rα (IL-15/sIL-15Rα) in the culture supernatants by CAR NK cells expressing the complexes of IL-15:IL-15Rα variants. The secretion of soluble IL-15 in the culture supernatants was determined in ELISA assays for the gene-modified NK cells derived from two healthy donors and the results from each donor are shown in (A) and (B), respectively. Mean values±SD are shown; n=3 technical replicates for each donor. The production of IL-15/sIL-15Rα heterodimers in the culture supernatants was determined in ELISA assays for the gene-modified NK cells derived from two healthy donors and the results from each donor are shown in (C) and (D), respectively. Mean values±SD are shown; n=3 technical replicates for each donor.

FIG. 4: Cytotoxicity of BDCA2 CAR NK cells expressing the complexes of IL-15:IL-15Rα variants against human K562 erythroleukemia cells, human RS4;11 B cell precursor acute lymphoblastic leukemia cells engineered to express GFP (RS4;11/GFP) or coexpress GFP and human BDCA2 (RS4;11/GFP/BDCA2). Mean values±SD are shown; n=3 technical replicates. Data were analyzed by two-tailed unpaired Student's t-test. ****, p<0.0001; **, p<0.01; ns: not significant (p>0.05).

FIG. 5: Anti-tumor activity of BDCA2 CAR NK cells expressing the complexes of IL-15:IL-15Rα variants against human RS4;11 B cell precursor acute lymphoblastic leukemia cells engineered to coexpress GFP and human BDCA2 (RS4;11/GFP/BDCA2) in repetitive tumor-challenge assays. Each panel represents the results of one donor. Mean values±SD are shown; n=3 technical replicates for each donor.

FIG. 6: In vitro proliferation of CAR NK cells expressing the complexes of IL-15:IL-15Rα variants shown in FIG. 5 in the absence of exogenous cytokine support.

FIG. 7: Fold expansions of CAR-negative and CAR-positive NK cells in the cell samples transduced with CAR and the complexes of IL-15:IL-15Rα variants shown in FIG. 6 Donor B.

FIG. 8: Cytokine secretion upon activation of CAR NK cells expressing the complexes of IL-15:IL-15Rα variants by tumor cells. The results from two healthy donors are shown in (A) and (B), respectively. Mean values±SD are shown; n=3 technical replicates for each donor.

FIG. 9: In vitro proliferation of CAR/IL15/215.2 NK cells in the absence of exogenous cytokine support with no evidence of dysregulated growth over time.

FIG. 10: Activity of CAR/IL15.215.2 NK cells against human RS4;11 B cell precursor acute lymphoblastic leukemia cells engineered to coexpress GFP and human BDCA2 (RS4;11/GFP/BDCA2). (A) Flow cytometric analysis of CAR expression on NK cells transduced with the CAR/IL15.215.2 construct. Untransduced (UTD) NK cells and NK cells only transduced with the CAR construct (CAR) served as controls. (B) Cytokine secretion upon activation of CAR/IL15.215.2 NK cells by RS4;11/GFP/BDCA2 cells.

FIG. 11: Activity of BDCA2 CAR T cells expressing the complexes of IL-15:IL-15Rα variants against human RS4;11 B cell precursor acute lymphoblastic leukemia cells engineered to coexpress GFP and human BDCA2 (RS4;11/GFP/BDCA2). (A) Flow cytometric analysis of CAR expression on CAR T cells expressing the complexes of IL-15:IL-15Rα variants. Untransduced (UTD) T cells and T cells only transduced with the CAR construct (CAR) served as controls. (B) The secretion of soluble IL-15 in the culture supernatants was determined in an ELISA assay for the gene-modified T cells shown in (A). Mean values±SD are shown; n=3 technical replicates. (C) The production of IL-15/sIL-15Rα heterodimer in the culture supernatants was determined in an ELISA assay for the gene-modified T cells. Mean values±SD are shown; n=3 technical replicates. (D) Anti-tumor activity of BDCA2 CAR T cells expressing the complexes of IL-15:IL-15Rα variants against human RS4;11 B cell precursor acute lymphoblastic leukemia cells engineered to coexpress GFP and human BDCA2 (RS4;11/GFP/BDCA2) in an repetitive tumor-challenge assay. Mean values±SD are shown; n=3 technical replicates.

FIG. 12: Natural cytotoxicity of NK cells expressing IL15/215.2 or IL15.215.2. (A) Flow cytometric analysis of CAR expression on NK cells transduced with the CAR/IL15, CAR/IL15/215.2 or CAR/IL15.215.2 construct. Untransduced (UTD) NK cells and NK cells transduced with the CAR construct only (CAR) served as controls. (B) Natural cytotoxicity of NK cells expressing the CAR/IL15, CAR/IL15/215.2 or CAR/IL15.215.2 construct against human K562 tumor cells engineered to express GFP (K562/GFP) in a repetitive tumor-challenge assay. Tumor growth was monitored by using the IncuCyte S3 system. Arrows indicate the time points of tumor challenges. Mean values±SD are shown; n=3 technical replicates.

FIG. 13: The advantage of fusing the intracellular CD2 domain to the C-terminus of the IL-15Rα receptor. (A) Lentiviral transfer plasmids encoding CD123 2.bb.z CAR/IL15/215 and CD123 bb.z CAR/IL15/215.2 under the transcriptional control of the human elongation factor 1 alpha (EF1α) promoter. Schematic representations of the CD123 2.bb.z CAR and IL15/215 constructs (B) the CD123 bb.z CAR and IL15/215.2 constructs (C). (D) Flow cytometric analysis of the expression of CAR on CD123 2.bb.z CAR/IL15/215 and CD123 bb.z CAR/IL15/215.2 NK cells. (E) Flow cytometric analysis of the CD123 expression on HEK293T and OCI-AML2/GFP cells. (F) Cytotoxicity of the gene-modified NK cells shown in (E) against human OCI-AML2 tumor cells engineered to express GFP (OCI-AML2/GFP) in a repetitive tumor-challenge assay. Tumor growth was monitored by using the IncuCyte S3 system. Arrows indicate the time points of tumor challenges. Mean values±SD are shown; n=3 technical replicates. (G) Cytokine secretion over time upon activation of the gene-modified NK cells shown in (F) by tumor cells. Mean values±SD are shown; n=3 technical replicates. Data were analyzed by two-tailed unpaired Student's t-test. ***, p<0.001; **, p<0.01; ns: not significant (p>0.05).

FIG. 14: The functionality of the IL-15:IL15Rα.CD2 complex when the IL-15 and IL15Rα.CD2 constructs are encoded by different nucleic acid sequences. (A) Lentiviral transfer plasmids encoding CD123 CAR, CD123 CAR/IL15 and LNGFR/215.2 under the transcriptional control of the human elongation factor 1 alpha (EF1α) promoter. (B) Flow cytometric analysis of the expression of CAR on UTD, CD123 CAR, CD123 CAR/IL15 and CD123 CAR/IL15+LNGFR/215.2 NK cells. The sequential transduction efficiency was further determined by the LNGFR and CAR expression for the NK cells transduced with the LNGFR/215.2 and CD123 CAR/IL15 constructs in two consecutive days. (C) Cytotoxicity of the gene-modified NK cells shown in (B) against human OCI-AML2 tumor cells engineered to express GFP (OCI-AML2/GFP) in a repetitive tumor-challenge assay. Tumor growth was monitored by using the IncuCyte S3 system. Arrows indicate the time points of tumor challenges. Mean values±SD are shown; n=3 technical replicates. (D) Cytokine secretion over time upon activation of the gene-modified NK cells shown in (C) by tumor cells. Mean values ±SD are shown; n=3 technical replicates. Data were analyzed by two-tailed unpaired Student's t-test. ****, p<0.0001; ***, p<0.001.

FIG. 15: In vitro proliferation of the gene-modified NK cells shown in FIG. 14 in the absence of exogenous cytokine support with no evidence of dysregulated growth over time.

### DETAILED DESCRIPTION OF THE INVENTION

In a first aspect the present invention provides a composition comprising
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2,
said second nucleic acid sequence comprising encoding IL-15.

Said nucleic acid sequences may be viral vectors such as retroviral vectors such as lentiviral vectors.

Said IL-15 may be the wildtype (wt) sequence of IL-15, e.g. human IL-15, or a functional variant thereof. Said IL-15 of composition A)I)b), A)II)i) and/or composition B) may comprise a signal peptide for secreting after expression in a cell. Said signal peptide may comprise the signal peptide of IL-15, CD33 or IL-2. Said IL-15 signal peptide may comprise SEQ ID:1. Said CD33 signal peptide may comprise SEQ ID:2. Said IL-2 signal peptide may comprise SEQ ID:3. Said IL-15 of composition A)I)b), A)II)i) and/or composition B) may comprise a propeptide after expression in a cell. Said propeptide may comprise SEQ ID NO:4. In some embodiments, said signal peptide and said propeptide may be cleaved from the mature IL-15 polypeptide.

The IL-15, i.e. mature IL-15 may comprise SEQ ID NO:5 (wt) or SEQ ID NO:6.

Said IL-15Rα of composition A)I)a)i) and/or composition B) may comprise a signal peptide for secreting after expression in a cell.

Said IL-15Rα of composition A)I)a)i) and/or composition B) may comprise SEQ ID NO:7.

Said IL-15Rα of composition A)II)iii) may comprise SEQ ID NO:8 (without signal peptide).

Said linker may be any linker that is able to link the IL-15 sequence with the IL-15Rα sequence and allows activation of the IL-15Rα upon binding of the IL-15 to the domain of IL-15Rα that binds the IL-15. Said linker may have a length between 4 to 50 amino acids, between 4 to 44 amino acids between 6 to 27 amino acids or between 10 to 20 amino acids. Said linker may have the nucleic acid sequence encoding SEQ ID NO:9.

The composition as disclosed herein, wherein said IL-15 of composition A)I)b) and composition B) comprises the signal peptide of IL-15 and the propeptide of IL-15, and wherein said IL-15 of composition A)II) i) has substituted said signal peptide of IL-15 and the propeptide of IL-15 by a second signal peptide such as the signal peptide of CD33 or the signal peptide of IL-2.

Said composition, wherein the nucleic acid sequence comprising encoding said IL-15Rα comprises SEQ ID NO:7, if said IL-15Rα is the IL-15Rα of A)I)a)i) or B) or wherein said nucleic acid comprising encoding said IL-15Rα comprises SEQ ID NO:8, if said IL-15Rα is the IL-15Rα of A)II)iii), and/or wherein the nucleic acid comprising encoding said intracellular signaling domain of CD2 comprises SEQ ID NO:10, and/or wherein the nucleic acid sequence comprising encoding said linker comprises SEQ ID NO:9, and/or wherein the nucleic acid comprising encoding said IL-15 comprises SEQ ID NO: 11, if said IL-15 is the IL-15 of A)I)b) or B) or wherein said nucleic acid comprising encoding said IL-15 comprises SEQ ID NO:12, if said IL-15 is the IL-15 of A)II)i).

The composition as disclosed herein, wherein said nucleic acid sequence of composition A)I) or composition A)II) additionally comprises a nucleic acid sequence encoding a transgene or wherein said composition of A)I) or said composition of A)II) comprises a further nucleic acid sequence encoding a transgene, or wherein said first nucleic acid sequence of B) or said second nucleic acid sequence of B) additionally comprise a nucleic acid sequence encoding a transgene.

Said composition, wherein said transgene may be a chimeric antigen receptor (CAR).

In another aspect the present invention provides a kit comprising
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2, said second nucleic acid sequence comprising encoding IL-15.

In a further aspect, the present invention provides an immune cell comprising
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2,
said second nucleic acid sequence comprising encoding IL-15.

The immune cell as disclosed herein, wherein the nucleic acid sequence comprising encoding said IL-15Rα comprises SEQ ID NO:7, if said IL-15Rα is the IL-15Rα of A)I)a)i) or B) or wherein said nucleic acid comprising encoding said IL-15Rα comprises SEQ ID NO:8, if said IL-15Rα is the IL-15Rα of A)II)iii), and/or wherein the nucleic acid comprising encoding said intracellular signaling domain of CD2 comprises SEQ ID NO:10, and/or wherein the nucleic acid sequence comprising encoding said linker comprises SEQ ID NO:9, and/or wherein the nucleic acid comprising encoding said IL-15 comprises SEQ ID NO:11, if said IL-15 is the IL-15 of A)I)b) or B) or wherein said nucleic acid comprising encoding said IL-15 comprises SEQ ID NO:12, if said IL-15 is the IL-15 of A)II)i).

The immune cell as disclosed herein, wherein said nucleic acid sequence of A)I) or A)II) additionally comprises a nucleic acid sequence encoding a transgene or wherein said immune cell comprises additionally to the nucleic acid sequence of A)I) or A)II) a further nucleic acid sequence encoding a transgene, or wherein said first nucleic acid sequence of B) or said second nucleic acid sequence of B) additionally comprise a nucleic acid sequence encoding a transgene.

The immune cell as disclosed herein, wherein said transgene is a chimeric antigen receptor (CAR).

The immune cell as disclosed herein, wherein said immune cell is an NK cell or a T cell.

The immune cell as disclosed herein for use in treatment of a disease. Said disease may be cancer, an infectious disease or an autoimmune disease.

The immune cell as disclosed herein for use in treatment of a disease or for use in immunotherapy.

The immune cell as disclosed herein for use in treatment of a disease, wherein said immune cell comprises a nucleic acid sequence encoding a transgene, wherein said transgene is a CAR specific for tumor associated antigen expressed on a cancer cell and wherein said disease is cancer.

In a further aspect, the present invention provides a pharmaceutical composition comprising 1) an immune cell comprising
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2,
said second nucleic acid sequence comprising encoding IL-15, and optionally
2) a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives.

In a further aspect the present invention provides a method of treating a disease in a subject comprising administering to said subject an immune cell comprising
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2,
said second nucleic acid sequence comprising encoding IL-15.

Said method, wherein said immune cell comprises a transgene, wherein said transgene is a CAR specific for an antigen such as a TAA expressed on the surface of a target cell such as a cancer cell.

Said method, wherein said nucleic acid sequence of composition A)I) or composition A)II) additionally comprises a nucleic acid sequence encoding a transgene or wherein said composition of A)I) or said composition of A)II) comprises a further nucleic acid sequence encoding a transgene, or wherein said first nucleic acid sequence of B) or said second nucleic acid sequence of B) additionally comprise a nucleic acid sequence encoding a transgene, wherein said additional nucleic acids sequences comprising said transgene may be a retroviral vector such as a lentiviral vector.

In a further aspect the present invention provides an in-vitro method for expanding genetically 25 engineered immune cells as disclosed herein comprising the step of expanding said genetically engineered immune cells without exogenous cytokine support in a culture medium that comprises said genetically engineered immune cells and non-genetically engineered immune cells. Said genetically engineered immune cells and said non-genetically engineered immune cells may be NK cells. Said genetically engineered immune cells and said non-genetically engineered immune cells may be T cells.

Said in-vitro method, wherein said expanding of said genetically engineered immune cells such as NK cells or T cells may be at least 1-fold, at least 2-fold, at least 3-fold, at least 4-fold, at least 5-fold, at least 6-fold, at least 7 fold, at least 8-fold, at least 9-fold, at least 10-fold, at least 11-fold, or at least 12-fold (as compared to the number of genetically engineered immune cells in said culture medium before said expansion step).

Said in-vitro method, wherein said expanding of said genetically engineered immune cells such as NK cells or T cells may be between 5-fold and 12-fold, between 6-fold and 12 fold, between 7-fold and 12 fold, between 8-fold and 12 fold (as compared to the number of genetically engineered immune cells in said culture medium before said expansion step), and wherein the expansion of total immune cells such as NK cells or T cells may be between 1-fold and 6-fold, between 1 fold and 5-fold, between 1-fold and 4-fold (as compared to the number of total immune cells in said culture medium before said expansion step), wherein total immune cells comprise said genetically engineered immune cells and said non-genetically engineered immune cells.

Said in-vitro method, wherein said expanding of said genetically engineered immune cells such as NK cells or T cells may be at least 10-fold (as compared to the number of genetically engineered immune cells in said culture medium before said expansion step), and wherein the expansion of total immune cells such as NK cells or T cells may at least 3-fold (as compared to the number of total immune cells in said culture medium before said expansion step), wherein total immune cells comprise said genetically engineered immune cells and said non-genetically engineered immune cells, or wherein said expanding of said genetically engineered immune cells such as NK cells or T cells may be at least 11-fold (as compared to the number of genetically engineered immune cells in said culture medium before said expansion step), and wherein the expansion of total immune cells such as NK cells or T cells may at least 3-fold (as compared to the number of total immune cells in said culture medium before said expansion step), wherein total immune cells comprise said genetically engineered immune cells and said non-genetically engineered immune cells.

Said in-vitro method, wherein said expanding of said genetically engineered immune cells such as NK cells or T cells may be at least 4 times faster or at least 5 times faster than the expansion of the non-engineered immune cells in said culture medium.

In a preferred embodiment of the invention an immune cell comprises
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2,
said second nucleic acid sequence comprising encoding IL-15,
wherein said nucleic acid sequence of A)I) or A)II) additionally comprises a nucleic acid sequence encoding a transgene or wherein said immune cell comprises additionally to the nucleic acid sequence of A)I) or A)II) a further nucleic acid sequence encoding a transgene, or wherein said first nucleic acid sequence of B) or said second nucleic acid sequence of B) additionally comprise a nucleic acid sequence encoding a transgene,
wherein said transgene is a chimeric antigen receptor (CAR),
wherein said immune cell is an NK cell or a T cell.

In another preferred embodiment of the invention an immune cell comprises
A) a nucleic acid sequence comprising encoding
I)
a) a fusion protein comprising from N-terminus to C-terminus
i) IL-15Rα and
ii) the intracellular signaling domain of CD2, and
b) IL-15
or
II) a fusion protein comprising from N-terminus to C-terminus
i) IL-15
ii) a linker
iii) IL-15Rα, and
iv) the intracellular signaling domain of CD2,
or
B) a first nucleic acid sequence and a second nucleic acid sequence, said first nucleic acid sequence comprising encoding a fusion protein comprising from N-terminus to C-terminus
i) IL-15Rα and
ii) the intracellular signaling domain of CD2,
said second nucleic acid sequence comprising encoding IL-15,
wherein said immune cell may be an NK cell, a T cell such as an αβ T cell or γδ T cell, a tumor infiltrating lymphocyte or a tumor reactive T cell, regulatory T (Treg) cells or an invariant NKT cell.

Such immune cells have endogenous cytotoxicity and are well-used to treat cancers.

Said immune cells may have endogenous immunoregulatory functions to treat autoimmune diseases.

In one embodiment of the invention the immune cell as disclosed herein may be further modified to reduce or eliminate expression of one or more endogenous genes, for example by disrupting an endogenous gene. The gene may be disrupted using gene editing techniques known in the art. Gene editing systems such as CRISPR/Cas systems, TALENs and zinc fingers can be used to generate double strand breaks, which, through gene repair mechanisms such as homology directed repair or non-homologous end joining (NHEJ), can be used to introduce mutations. NHEJ after resection of the ends of the break, or improper end joining, can be used to introduce deletions and/or disruptions of a gene.

In one embodiment of the invention the immune cell as disclosed herein may be further modified to express a exogenous gene under the control of endogenous promotor that are sensitive to immune cell activation by using said gene editing systems.

In one embodiment of the invention the immune cell as disclosed herein may be further modified by combining knockout of one or more endogenous genes subsequently followed by knockin of transgenes driven under the control of the endogenous promotor by using said gene editing systems.

In one embodiment of the invention the immune cells as disclosed herein may express a CAR specific for an antigen for use in treatment of a disease associated with a target cell of a subject suffering from said disease, the disease may be e.g. cancer and the target cell a cancerous cell.

Immune cells, e.g. T cells or NK cells of a subject may be isolated. The subject may e.g. suffer from said cancer or may be a healthy subject. These cells are genetically modified in vitro to express an IL-15:IL-15Rα-CD2 construct as disclosed herein and said CAR. These engineered cells may be activated and expanded in vitro or in vivo. In a cellular therapy these engineered cells are infused to a recipient in need thereof. These cells may be a pharmaceutical composition (said cell plus pharmaceutical acceptable carrier). The infused cells may be e.g. able to kill (or at least stop growth of) cancerous cells in the recipient. The recipient may be the same subject from which the cells was obtained (autologous cell therapy) or may be from another subject of the same species (allogeneic cell therapy).

The immune cells, preferentially T cells or NK cells engineered to express the IL-15:IL-15Rα-CD2 construct and the CAR may be administered either alone, or as a pharmaceutical composition in combination with diluents and/or with other components such as IL-2 or other cytokines or cell populations. Briefly, pharmaceutical compositions of the present invention may comprise a cell population of genetically modified cells (a plurality of immune cells) as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives.

Preferentially, the compositions of the present invention are formulated for intravenous administration. The administration of cell compositions to the subject may be carried out in any convenient manner known in the art.

Pharmaceutical compositions of the present invention may be administered in a manner appropriate to the disease to be treated. Appropriate dosages may be determined by clinical trials. But the quantity and frequency of administration will also be determined and influenced by such factors as the condition of the patient, and the type and severity of the patient's disease.

A pharmaceutical composition comprising the immune cells, preferentially T cells or NK cells as disclosed herein may be administered at a dosage of 10⁴ to 10⁹ cells/kg body weight, preferably 10⁵ to 10⁶ cells/kg body weight. The cell compositions may also be administered several times at these dosages. The compositions of cells may be injected e.g. directly into a tumor, lymph node, or site of infection.

The genetically engineered immune cells may be activated and expanded to therapeutic effective amounts using methods known in the art.

The immune cells of the invention may be used in combination with e.g. chemotherapy, radiation, immunosuppressive agents, antibodies or antibody therapies.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

In general, a CAR may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell.

Such a CAR may be referred to as “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target. The tag may be e.g. dextran or a hapten such as biotin or fluorescein isothiocyanate (FITC) or phycoerythrin (PE) or thiamin, but the tag may also be a peptide sequence e.g. chemically or recombinantly coupled to the polypeptide part of the tagged polypeptide. The tag may also be streptavidin. The tag portion of the tagged polypeptide is only constrained by being a molecule that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv, respectively.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs of the invention may comprise one or more antigen binding domains (e.g. a tandem CAR). Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR, otherwise it is indicated explicitly as an inhibitory CAR (iCAR)). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, DAP12, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiments, the endodomain may contain a primary cytoplasmic signaling domains or a co-stimulatory region, but not both.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adapter molecules could be designed for tumor specificity and would provide options for altering specificity post-adoptive transfer, key for situations of selection pressure and antigen escape.

The CARs may be designed to comprise any portion or part of the above-mentioned domains as described herein in any order and/or combination resulting in a functional CAR, i.e. a CAR that mediated an immune effector response of the immune effector cell that expresses the CAR as disclosed herein.

The term “tagged polypeptide” as used herein refers to a polypeptide that has bound thereto directly or indirectly at least one additional component, i.e. the tag. The tagged polypeptide as used herein is able to bind an antigen expressed on a target cell. The polypeptide may be an antibody or antigen binding fragment thereof that binds to an antigen expressed on the surface of a target cell such as a tumor associated antigen on a cancer cell. The polypeptide of the tagged polypeptide alternatively may be a cytokine or a growth factor or another soluble polypeptide that is capable of binding to an antigen of a target cell.

The terms “adapter” or “adapter molecule” or “tagged polypeptide” as used herein may be used interchangeably.

The tag may be e.g. a hapten or dextran and the hapten or dextran may be bound by the antigen binding domain of the polypeptide, e.g. a CAR, comprising an antigen binding domain specific for the tag.

Haptens such as e.g. FITC, biotin, PE, streptavidin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

But the tag may also be a peptide sequence e.g. chemically or recombinantly coupled to the polypeptide part of the tagged polypeptide. The peptide may be selected from the group consisting of c-Myc-tag, Strep-Tag, Flag-Tag, and Polyhistidine-tag. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

An “interleukin-15 protein” or “IL-15” as referred to herein may include any of the recombinant or naturally-occurring forms of the IL-15 or variants or homologs thereof that maintain IL-15 protein activity, i.e. they may be functional variants of IL-15 (e.g. within at least 50%, 80%, 90%, 95%, 96%, 97%, 98%, 99% or 100% activity compared to IL-15). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15. In embodiments, the IL-15 protein may be substantially identical to the protein identified by the UniProt reference number P40933 or a variant or homolog having substantial identity thereto.

Preferentially, the IL-15 is human IL-15.

Said IL-15Rα may be the wildtype sequence of IL-15Rα or a functional fragment thereof comprising the transmembrane domain of IL-15Rα and a domain that can bind to IL-15 such as e.g. IL-15Rα Sushi domain (IL-15Rα Su).

An “interleukin-15 receptor subunit alpha protein” or “IL-15Rα” as referred to herein may include any of the recombinant or naturally-occurring forms of the IL-15Rα or variants or homologs thereof that maintain IL-15Rα protein activity, i.e. they are functional variants of IL-15Rα (e.g. within at least 50%, 80%, 90%, 95%, 96%, 97%, 98%, 99% or 100% activity compared to IL-15Rα). In embodiments, the variants or homologs have atleast 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15Rα. In embodiments, the IL-15Rα may be substantially identical to the protein identified by the UniProt reference number Q13261 or a variant or homolog having substantial identity thereto.

As used herein, “domain” refers to a conserved portion of a protein that functions and exists independently of the rest of the protein sequence. A domain may form a stable, three-dimensional structure that exists as a functional unit independent of the remaining protein. For example, the IL-15Rα Su domain is the portion of IL-15Rα that retains the IL-15 binding activity.

The term “sushi domain” as used herein refers to a common motif in proteins comprising a beta-sandwich arrangement. Sushi domains are common in protein-protein interactions, and typically include four cysteines forming two disulfide bonds in a 1-3 and 2-4 pattern. For example, the region of IL-15Rα that binds IL-15 includes a sushi domain.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”).

Examples of antigen binding fragments include Fab (fragment antigen binding), scFv (single chain fragment variable), single domain antibodies (nanobodies), diabodies, dsFv, Fab′, diabodies, single-chain antibody molecules, and multispecific antibodies formed from antibody fragments. The antibody or antibody fragment may be human, fully human, humanized, human engineered, non-human, and/or chimeric. The non-human antibody or antibody fragment may be humanized to reduce immunogenicity to humans, while retaining the specificity and affinity of the parental non-human antibody. Chimeric antibodies may refer to antibodies created through the joining of two or more antibody genes which originally encoded for separate antibodies.

The terms “having specificity for”, “specifically binds” or “specific for” with respect to an antigen-binding domain of an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain is specific. An antigen-binding domain that specifically binds to an antigen may bind also to different allelic forms of the antigen (allelic variants, splice variants, isoforms etc.). This cross reactivity is not contrary to the definition of that antigen-binding domain is specific.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed e.g. on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “soluble antigen” as used herein refers to an antigen that is not immobilized on surfaces such as beads or cell membranes.

The terms “immune cell” or “immune effector cell” may be used interchangeably and refer to a cell that may be part of the immune system and executes a particular effector function such as alpha-beta T cells, NK cells, NKT cells, B cells, innate lymphoid cells (ILC), cytokine induced killer (CIK) cells, lymphokine activated killer (LAK) cells, gamma-delta T cells, regulatory T cells (Treg), monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells. Most preferred immune effector cells are T cells and NK cells. Tumor infiltrating lymphocytes (TTLs) are T cells that have moved from the blood of a subject into a tumor. These TTLs may be removed from a patient's tumor by methods well known in the art, e.g. enzymatic and mechanic tumor disruption followed by density centrifugation and/or cell marker specific enrichment. TTLs are genetically engineered as disclosed herein, and then given back to the patient. “Effector function” means a specialized function of a cell, e.g. in a T cell an effector function may be cytolytic activity or helper activity including the secretion of cytokines.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. For example, T cells or NK cells that have a natural or genetically engineered reactivity to a patient's cancer are generated in-vitro and then transferred back into the cancer patient. Then the immunotherapy is referred to as “CAR cell immunotherapy” or in case of use of T cells only as “CAR T cell therapy” or “CAR T cell immunotherapy”.

The term “treatment” as used herein means to reduce the frequency or severity of at least one sign or symptom of a disease.

The term “autologous” as used herein refers to any material derived from the same subject to who it is later re-introduced.

The term “allogeneic” as used herein refers to any material derived from a different subject of the same species as the subject to who the material is re-introduced.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from an allergic disease or from an infectious disease or from graft rejection.

The term “expression” as used herein is defined as the transcription of a particular nucleotide sequence into RNA and optionally subsequent translation of said RNA into a polypeptide sequence or a protein.

A fusion protein or chimeric protein is a protein created through the joining of two or more genes that originally coded for separate proteins. Translation of this fusion gene results in a single polypeptide with functional properties derived from each of the original proteins.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refer to the fact that cells, preferentially T cells or NK cells can be manipulated by recombinant methods well known in the art to express stably or transiently peptides or proteins which are not expressed in these cells in the natural state.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The terms “nucleic acid”, “nucleic acid sequence” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means. A kit may comprise a container with components within the container. Such containers may be e.g. boxes, bottles, vials, tubes, bags, pouches, blister packs, or other suitable container forms known in the art. Such containers may be made of plastic, glass, laminated paper, metal foil, or other materials suitable for holding components therein. The kit further may comprise written directions for using the components of the kits.

### EXAMPLES

The following examples are intended for a more detailed explanation of the invention but without restricting the invention to these examples.

### Example 1: Design and Generation of Lentiviral Vectors Encoding CARs and the Complexes of IL-15:IL-15Rα Variants

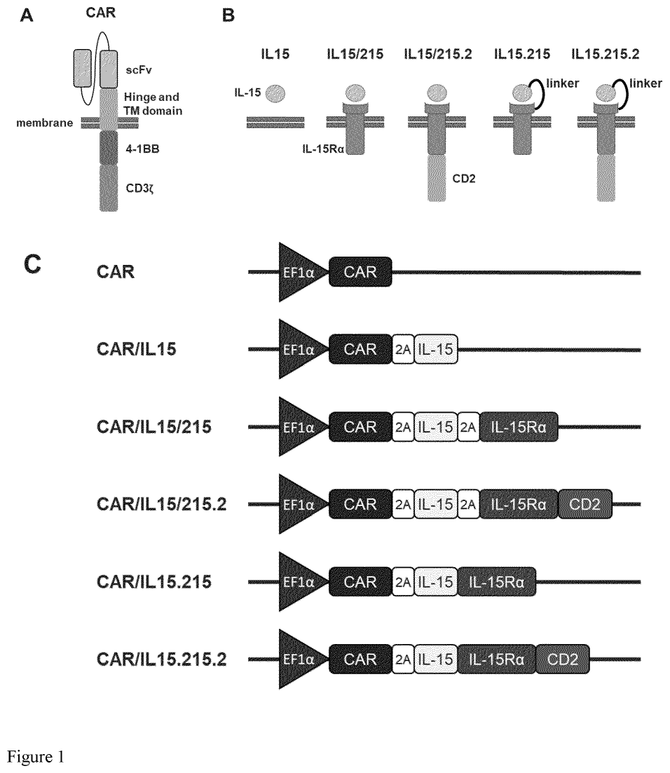

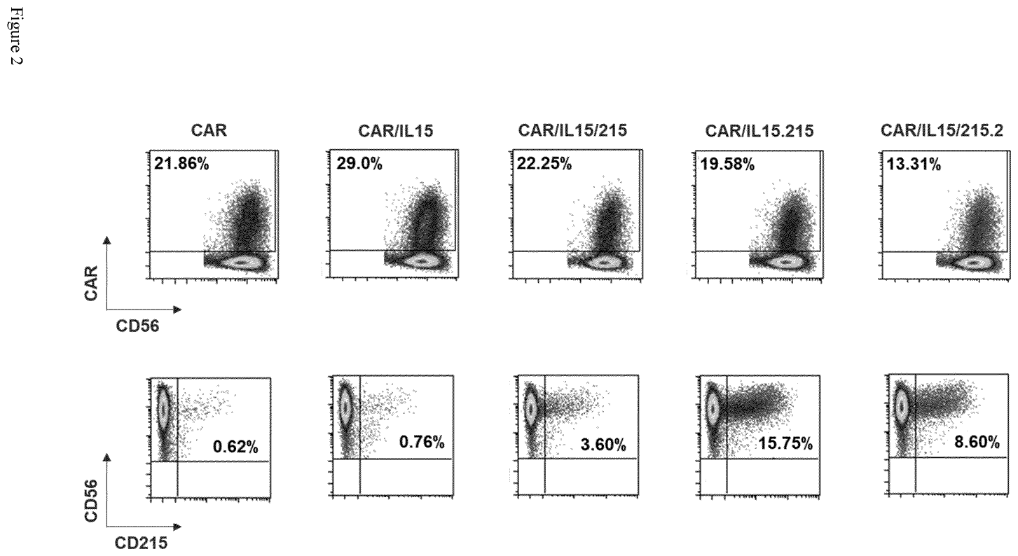

A blood dendritic cells antigen 2 (BDCA2)-specific CAR as a gene of interest and different complexes of IL-15:IL-15Rα variants were designed in silico. The BDCA2 CAR sequence consists of the human granulocyte macrophage colony stimulating factor receptor alpha subunit (GM-CSFRa) signal peptide, a BDCA2-specific single chain Fv (scFv) antibody fragment, the CD8α hinge and transmembrane (TM) domain, followed by the intracellular (IC) domains of 4-1BB and CD3ζ (FIG. 1A).

Native IL-15 (NM_000585.4) including its signal peptide and propeptide was used to generate the IL15 construct. The IL15/215 construct encoding sequence was generated by linking native IL-15 to IL-15Rα (also known as CD215; NM_002189.3) with a thosea asigna virus 2A (T2A) self-cleaving peptide sequence. The IL15/215.2 construct was created by fusing the CD2 intracellular domain to the C-terminus of IL15/215 construct. The 2A peptide sequence was replaced by a 26 amino acid linker [SEQ ID NO:9] and the IL-15 signal peptide and propeptide in the IL15/215 and IL15/215.2 constructs were substituted by the human CD33 signal peptide, resulting in the membrane-bound constructs IL15.215 and IL15.215.2, respectively (FIG. 1B). The cassettes encoding CARs and the complexes of IL-15:IL-15Rα variants were then cloned into a self-inactivating, third generation lentiviral transfer plasmid backbone under the control of a human elongation factor 1 alpha (EF1α) promoter (FIG. 1C). CARs and the complexes of IL-15:IL-15Rα variants were separated by using porcine teschovirus-1 2A (P2A) self-cleaving peptide sequences. Baboon envelope (BaEV) pseudotyped lentiviral vector (LV) containing supernatants were produced by transient transfection of HEK 293T cells. LV-containing supernatants are stored at −80° C. before use.

### Example 2: Generation of CAR-Transduced NK Cells Expressing the Complexes of IL-15:IL-15Rα Variants

Peripheral blood mononuclear cells (PBMC) were isolated from healthy donors by Pancoll (Pan biotech, Aidenbach, Germany) density gradient centrifugation. Primary human NK cells were enriched from PBMCs with CD3-positive cell depletion followed by CD56-positive cell enrichment using antibody coated microbeads (Miltenyi Biotec, Bergisch Gladbach, Germany). Primary NK cells were cultured in NK cell culture medium containing NK MACS medium (Miltenyi Biotec), supplemented with 5% heat inactivated AB serum (Access Cell Culture, Vista, CA, USA), 500 IU/mL human IL-2 and 140 IU/mL human IL-15 (Miltenyi Biotec). To transduce NK cells, purified NK cells were stimulated with NK cell culture medium additionally supplemented with 80 ng/ml of a human IL-1 family cytokine (Miltenyi Biotec) after isolation (Day 0). At day 2, NK cells were transduced with BaEV-LV encoding BDCA2 CAR and the complexes of IL-15:IL-15Rα variants in the presence of 2.5 g/mL Vectofusin-1 for 24 h, after 2 h spinoculation at 400×g, 32° C. The transduced cells were further cultured in fresh, complete NK cell culture medium. Half of the conditioned culture medium was exchanged in every 2-3 days. The surface expression of CAR and IL-15Rα on NK cells were determined at day 14 after isolation with flow cytometric analysis. CAR expression was detected by using biotinylated human recombinant BDCA2-Fc protein followed by VioBright515-conjugated anti-biotin antibody (Miltenyi Biotec). IL-15Rα expression was detected by using APC-conjugated anti-IL-15Rα antibody (Clone JM7A4, Biolegend, Amsterdam, The Netherlands). A representative experiment is shown in FIG. 2.

The data summarized in Example 2 demonstrate that CARs as genes of interest and the complexes of IL-15:IL-15Rα variants can be expressed by gene-modified cells.

### Example 3: Production of IL-15 and IL-15/sIL-15Rα Heterodimer by Gene-Modified Immune Cells

Next, we assessed the secretion of soluble IL-15 by gene-modified cells. CAR NK cells expressing the complexes of IL-15:IL-15Rα variants were generated as described in Example 2. After 14 days of culture, CAR-expressing NK cells were counted by flow cytometry and washed for 3 times by centrifugation with NK MACS medium only supplemented with 5% heat inactivated AB serum. The percentage of CAR-positive NK cells in all the samples were adjusted by adding unmodified NK cells, according to the sample with the lowest transduction efficiency. The same number of CAR-positive NK cells was seeded for all the samples in NK cell culture medium without exogenous cytokine support and further cultivated for 2 days. Supernatants were then collected and soluble IL-15 was determined by using ELISA MAX™ Deluxe Set Human IL-15 kit (Biolegend), according to the manufacturers' instructions.

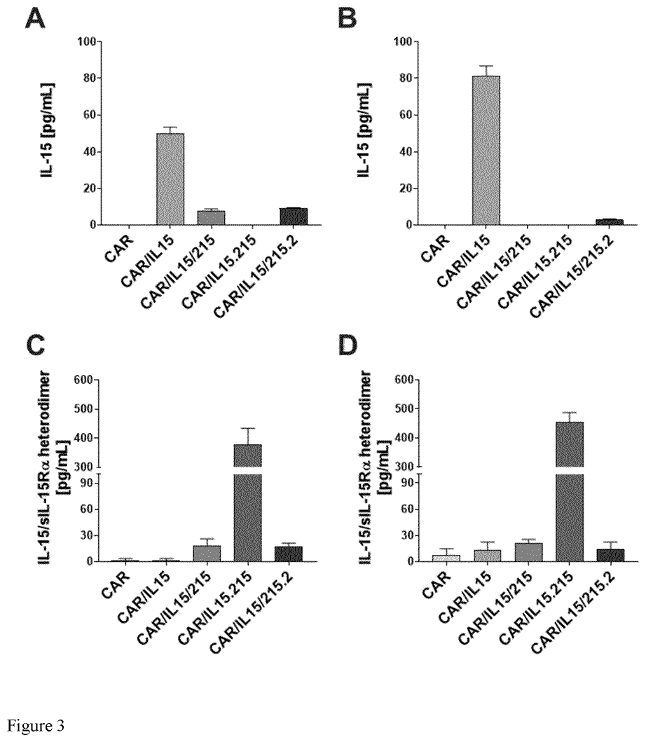

The values of IL-15 in the supernatants collected from NK cells transduced with the CAR and CAR/IL15.215 constructs were below the detection limit (4 pg/mL) of the ELISA kit from Biolegend for both donors (FIGS. 3A and B). However, NK cells transduced with the CAR/IL15 construct secreted higher level of soluble IL-15 into the culture supernatants, resulting in mean 49.74 pg/mL and mean 81.27 pg/mL in the experiments with two donors, respectively (FIGS. 3A and B). Of note, soluble IL-15 could only be detected at very low levels in the supernatants collected from NK cells transduced with the CAR/IL15/215 (mean 7.52 pg/mL) and CAR/IL15/215.2 (mean 9.11 pg/mL) constructs for one donor (FIG. 3A), but were undetectable for the other donor (FIG. 3B).

It has been shown that soluble form of IL-15Rα (sTL-15Rα) naturally released by proteolytic cleavage of the membrane-bound receptor (Mortier et al., 2004 J. Immunol. 173, 1681-1688). Circulating IL-15 has been reported to associate with sIL-15Rα to form heterodimers in human serum (Bergamaschi et al., 2012, Blood 120, e1-e8). Therefore, we assessed the presence of IL-15/sIL-15Rα heterodimers in the culture supernatant of the gene-modified immune cells. Supernatants from CAR NK cells expressing the complexes of IL-15:IL-15Rα variants were prepared as previously described in Example 3. Human IL-15/sIL-15Rα heterodimers were determined by Human IL-15/IL-15R alpha Complex DuoSet ELISA kit (R&D Systems, Minneapolis, MN, USA), according to the manufacturers' instructions.

IL-15/sIL-15Rα heterodimers were undetectable in the supernatants derived from NK cells transduced with the CAR and CAR/IL15 constructs for one donor (FIG. 3C), but could be found at low levels for the other donor, resulting in mean 7.05 pg/mL and mean 12.77 pg/mL, respectively (FIG. 3D). Moderately higher levels of IL-15/sIL-15Rα heterodimers were detected in the supernatants collected from NK cells transduced with the CAR/IL15/215 and CAR/IL15/215.2 constructs for both donors, ranging from 18.50 pg/mL to 21.05 pg/mL and from 14.03 pg/mL to 17.21 pg/mL, respectively (FIGS. 3C and D). Intriguingly, the highest IL-15/sIL-15Rα heterodimer levels were detected in the supernatants prepared from CAR/IL15.215 NK cells for both donors, resulting in mean 377.67 pg/mL and 454.33 pg/mL of IL-15/sIL-15Rα heterodimers for both donors, respectively (FIGS. 3C and D). It may be arisen from proteolytic shedding of IL15.215, which is a membrane-bound recombinant receptor fusing IL-15 to IL-15Rα via a 26 aa linker (FIG. 1).

The data summarized in Example 3 demonstrate that only negligible to low amount soluble IL-15 and IL-15/sIL-15Rα heterodimers were released by NK cells transduced with the CAR/IL15/215.2 construct to the culture supernatants, indicating the potential low risk of systemic inflammatory response caused by free IL-15 and IL-15/sIL-15Rα heterodimers when applying CAR/IL15/215.2-transduced cells in the clinic.

### Example 4: Expression of the Complexes of IL-15:IL-15Rα Variants does not Impair the Specificity of CAR-Mediated Cytotoxicity of Gene-Modified Immune Cells

To assess the CAR-mediated cytotoxicity of gene-modified NK cells, we coincubated NK cells with RS4;11 cells engineered to express GFP (RS4;11/GFP) or GFP and human BDCA2 (RS4;11/GFP/BDCA2), respectively. The natural cytotoxicity of gene-modified NK cells were determined by coincubating NK cells with CellTrace Violet (CTV)-labelled K562 cells, which are known as NK cell-sensitive targets due to lacking the expression of major histocompatibility complex class I (MHC I) molecules. CARNK cells expressing the complexes of IL-15:IL-15Rα variants were generated as described in Example 2. The percentage of CAR-positive NK cells in all the samples were adjusted by adding unmodified NK cells, according to the sample with the lowest transduction efficiency. NK cells and target cells were coincubated for 24 h with an effector to target (E:T) ratio of 1:2, which was calculated based on the number of transduced NK cells. Target cells without NK cells were prepared to serve as controls. At the end of cytotoxicity assays, propidium iodide (PI, Miltenyi Biotec) was added to each sample to quantify the number of viable target cells by using a MACSQuant Analyzer 10 flow cytometer. The antitumor efficacy was calculated with the formula shown below.

$$( 1 - \frac{number of target cells in the well}{\begin{matrix}average of the number of \\ the targets cells cultivated alone\end{matrix}} ) * 100 \%$$

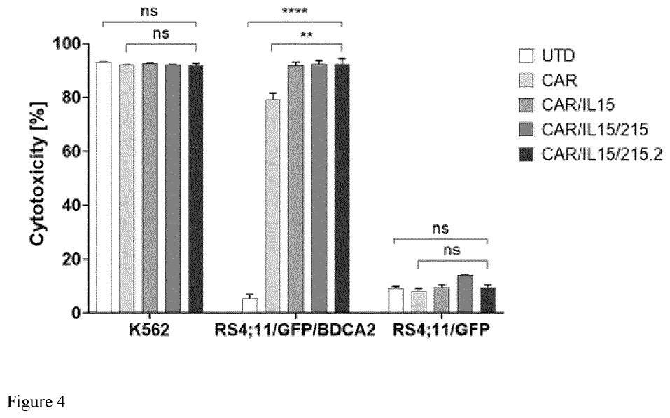

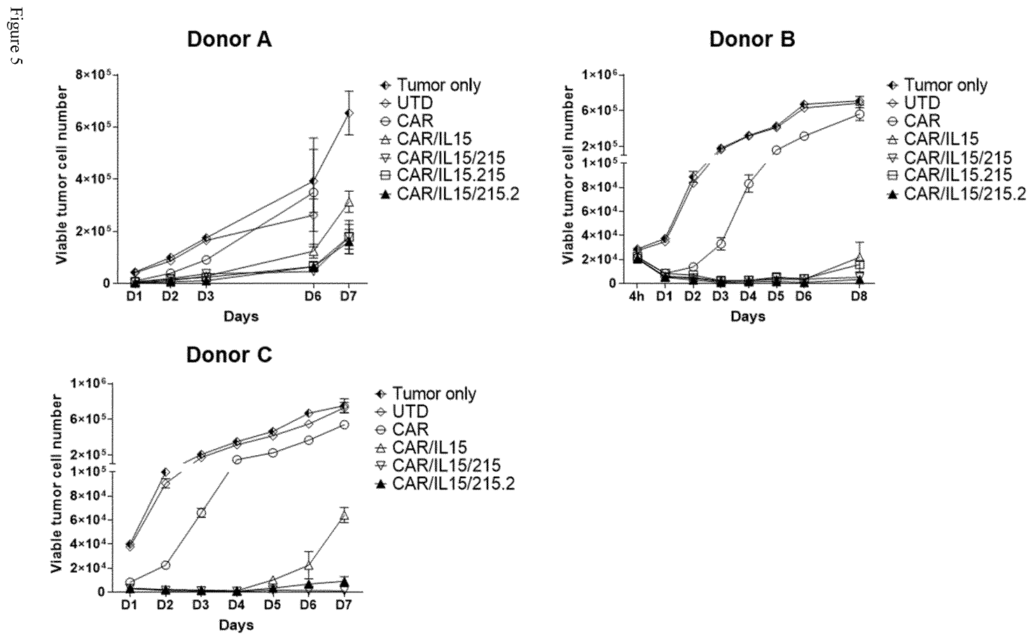

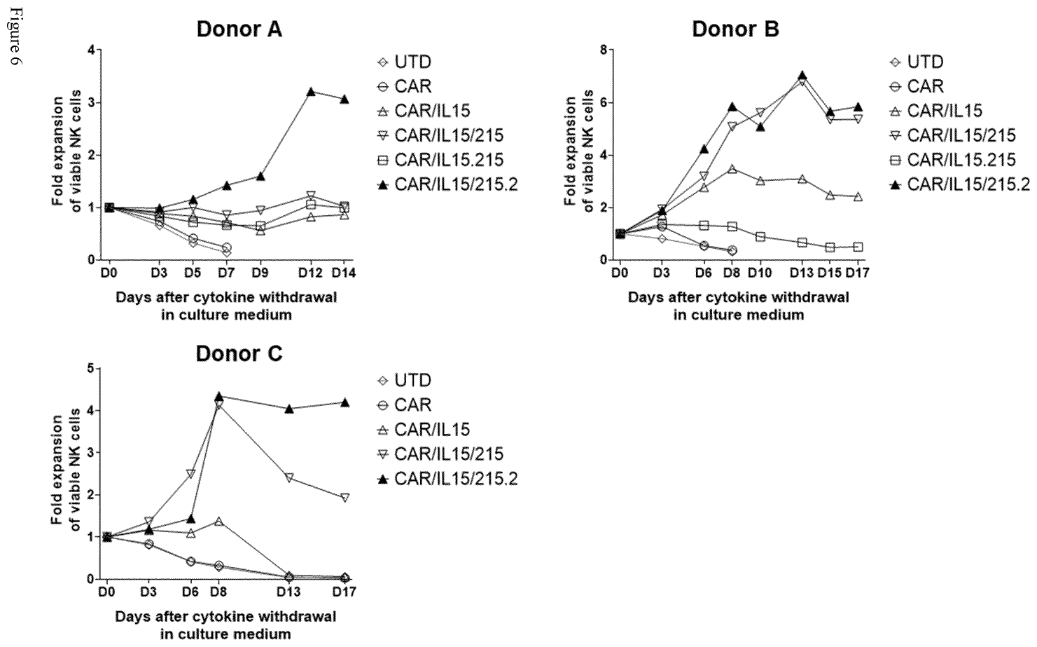

High anti-tumor activity against K562 target cells was shown for all the NK cell samples, whereas RS4;11/GFP/BDCA2 tumor cells, which are otherwise resistant to NK cell natural cytotoxicity, could only be efficiently lysed by NK cells expressing BDCA2-specific CARs. Meanwhile, RS4;11/GFP tumor cells remained largely resistant towards CAR/IL15/215.2 NK cells, resulting in no significant difference in tumor cell lysis compared with untransduced (UTD) NK cells or CAR NK cells. Intriguingly, the expression of IL15/215.2 construct significantly enhanced CAR-mediated NK cell cytotoxicity against BDCA2-expressing RS4;11 cells, compared with NK cells only transduced with BDCA2 CARs (FIG. 4).

The data summarized in Example 4 demonstrate that the expression of IL15/215.2 does not impair the killing specificity of CAR NK cells, however, it can enhance CAR-mediated cytotoxicity of CAR-expressing immune cells.

### Example 5: CAR-Transduced NK Cells Expressing IL15/215.2 Display Sustained Anti-Tumor Activity

To assess the potential of IL15/215.2 on maintaining the functionality of gene-modified NK cells in the absence of exogenous cytokine support, BDCA2 CARNK cells expressing different complexes of IL-15:IL-15Rα variants were generated according to the method described in Example 2. The percentage of CAR-positive NK cells in all the samples were adjusted by adding unmodified NK cells, according to the sample with the lowest transduction efficiency. The generated gene-modified NK cells were coincubated with 3×10⁴ RS4;11/GFP/BDCA2 tumor cells at an initial E:T ratio of 1:2, which was calculated based on the number of transduced NK cells. The gene-modified NK cells were then repeatedly challenged for 7-8 days with 3×10⁴ fresh RS4;11/GFP/BDCA2 tumor cells in every 24 h. Gene-modified NK cells and tumor cells were coincubated in RPMI1640 medium supplemented with 10% fetal bovine serum (FBS) and 2 mM L-Glutamine without additional cytokines in favor of tumor cell growth. At the time indicated for each experiment in FIG. 5, dead cells were excluded by using PI (Miltenyi Biotec) and the numbers of remaining viable target tumor cells were counted by using a MACSQuant Analyzer 10 flow cytometer. Target cells incubated without NK cells and with UTD NK cells were prepared to serve as controls.

Tumor cells rapidly grew in the samples coincubated without NK cells or with UTD NK cells in all three experiments using NK cells derived from different healthy donors. CAR/IL15 NK cells initially inhibited tumor outgrowth until tumor cells started to grow out at day 3-6 showed in the different experiments. CAR/IL15.215 NK cells have been tested in donor A and B, resulting in improved tumor control compared with CAR/IL15 NK cells. Of note, both CAR/IL15/215 and CAR/IL15/215.2 NK cells displayed the most efficient long-term tumor control in all the experiments (FIG. 5).

The data summarized in Example 5 demonstrate that the expression of IL15/215.2 in CAR-expressing NK cells can sustain their cytotoxicity against tumor cells in the absence of exogenous cytokine support, thereby promoting their continued anti-tumor response.

### Example 6: CAR-Transduced NK Cells Expressing IL15/215.2 Display Enhanced Proliferation Capacity

Next, we investigated the functionality of IL15/215.2 in supporting immune cell survival and proliferation. The gene-modified NK cells shown in Example 5 were washed for 3 times by centrifugation with NK MACS medium only supplemented with 5% heat inactivated AB serum, and 1×10⁶ total NK cells were seeded in NK cell culture medium without exogeneous cytokines. NK cell growth was monitored for 14-17 days. Dead cells were excluded with PI and viable NK cell numbers were counted by using a MACSQuant Analyzer 10 flow cytometer. NK cells transduced with the CAR/IL15/215.2 construct exhibited the most efficient expansion for all three donors after withdrawal of exogenous cytokines from the NK cell culture medium, while UTD and CAR NK cells rapidly declined in number over the course of the experiments. Both CAR/IL15 and CAR/IL15.215 NK cells could maintain their survival with moderate expansion folds ranging from 0.89 to 3.48 and from 1.06 to 1.35 in the experiments, respectively. It is similar to the previous observations (Hurton et al., 2016, Proc. Natl. Acad. Sci. 113, E7788-E7797; Liu et al., 2018, Leukemia 32, 520-531). Compared with CAR/IL15/215.2 NK cells, CAR/IL15/215 NK cells showed comparable growth in Donor B, but either no marked expansion or earlier cell number decrease in the other two donors, indicating a strong donor-dependent effect underlay for IL15/215 (FIG. 6). Of note, CD2 and its natural ligand CD58 are also expressed on the surface of NK cells and their expression levels increase 4- to 6-fold after in vitro incubation with IL-2 (Robertson et al. 1990, J. Immunol. 145, 3194-3201). It is thereby likely the endogenous CD2 signaling in NK cells can be induced by neighboring NK cells expressing CD58. However, the most efficient and robust expansions were only observed for CAR/IL15/215.2 NK cells, indicating the superior functionality of IL15/215.2 by integrating the intracellular signaling domain of CD2 into the IL-15:IL-15Rα complex. The data summarized in Example 6 demonstrate that IL15/215.2 can endow CAR-transduced immune cells with enhanced proliferation and survival capacity.

### Example 7: Expansion of Gene-Modified NK Cells Expressing the Complexes of IL-15:IL-15Rα Variants in the Absence of Exogenous Cytokine Support

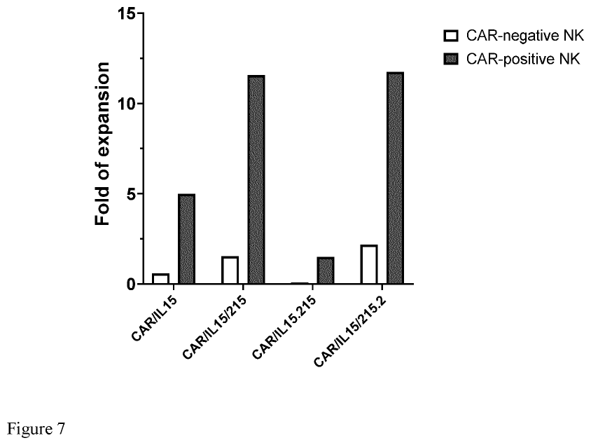

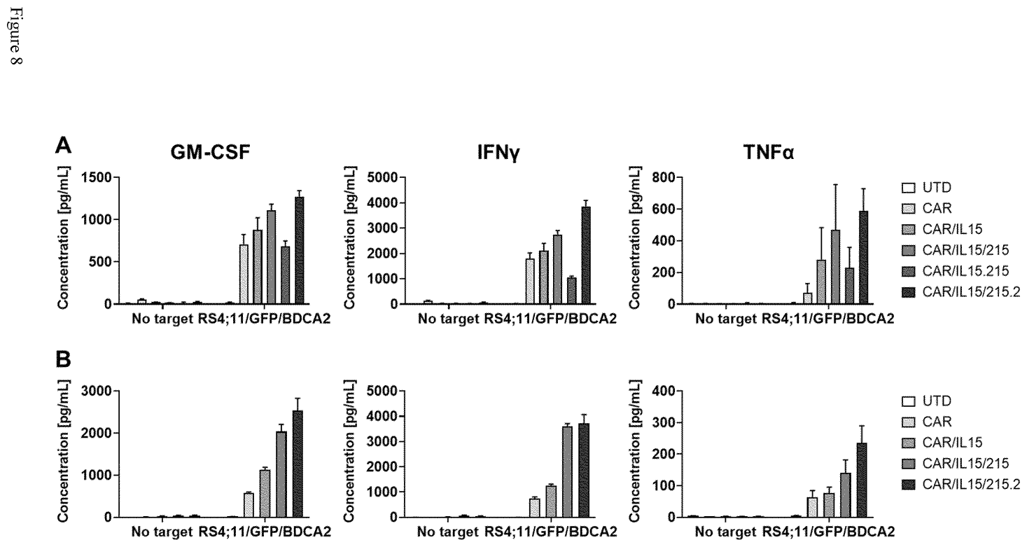

The expansion of CAR-positive and CAR-negative NK cells from donor B shown in Example 6 was determined at the end point of the assay. CAR expression was detected by using biotinylated human recombinant BDCA2-Fc protein followed by VioBright515-conjugated anti-biotin antibody (Miltenyi Biotec) by using a MACSQuant Analyzer 10 flow cytometer. While no viable NK cells could be detected in the end of the assay for UTD NK cells and CAR NK cells, the CAR-positive NK cells expanded in all the samples transduced with CARs and the complexes of IL-15:IL-15Rα variants, resulting in 5.0-, 11.57-, 1.5- and 11.73-fold expansion for CAR/IL15, CAR/IL15/215, CAR/IL15.215 and CAR/IL15/215.2 NK cells, respectively (FIG. 7). Unlike the samples transduced with the CAR/IL15 and CAR/IL15.215 constructs showing no expansion of CAR-negative NK cells, the CAR-negative NK cells displayed 1.52- and 2.17-fold expansion in the samples transduced with the CAR/IL15/215 and CAR/IL15/215.2 constructs, respectively.

The data summarized in Example 7 demonstrate that gene-modified immune cells expressing IL15/215.2 can be effectively expanded in the absence of exogenous cytokine support.

### Example 8: IL15/215.2 Enhances Proinflammatory Cytokine Secretion in CAR-Transduced NK Cells Upon CAR-Mediated Activation

Cytokine production of gene modified immune cells were further evaluated. The gene-modified NK cells from donor A and C shown in Example 5 were coincubated at an E:T ratio of 1:1 for 24 h with RS4;11 tumor cells engineered to express GFP and human BDCA2 (RS4;11/GFP/BDCA2). UTD NK cells and NK cells without tumor cell stimulation were included as controls. Supernatants were collected and the levels of cytokines were measured using a MACSPlex Cytokine 12 Kit, human (Miltenyi Biotec), according to the manufacturer's instructions. Coculturing with RS4;11/GFP/BDCA2 tumor cells markedly upregulated the secretion of proinflammatory cytokines, such as granulocyte-macrophage colony-stimulating factor (GM-CSF), interferon γ (IFNγ) and tumor necrosis factor α (TNFα), for CAR-expressing NK cells, but not for UTD NK cells, while no marked cytokine secretion were observed for the gene-modified NK cells without tumor target cell stimulation (FIG. 8). It indicates that ectopic expression of the complexes of IL-15:IL-15Rα variants alone do not induce cytokine expression in NK cells. The highest cytokine levels were observed for CAR/IL15/215.2 NK cells in the samples from both donor A (FIG. 8A) and donor C (FIG. 8B).

The data summarized in Example 8 demonstrate that the expression of IL15/215.2 can increase proinflammatory cytokine expression in gene-modified immune cells, thereby enhancing their potency in therapy.

### Example 9: Ectopic Expression of IL15/215.2 does not Induce Autonomous or Dysregulated Growth in CAR-Modified NK Cells

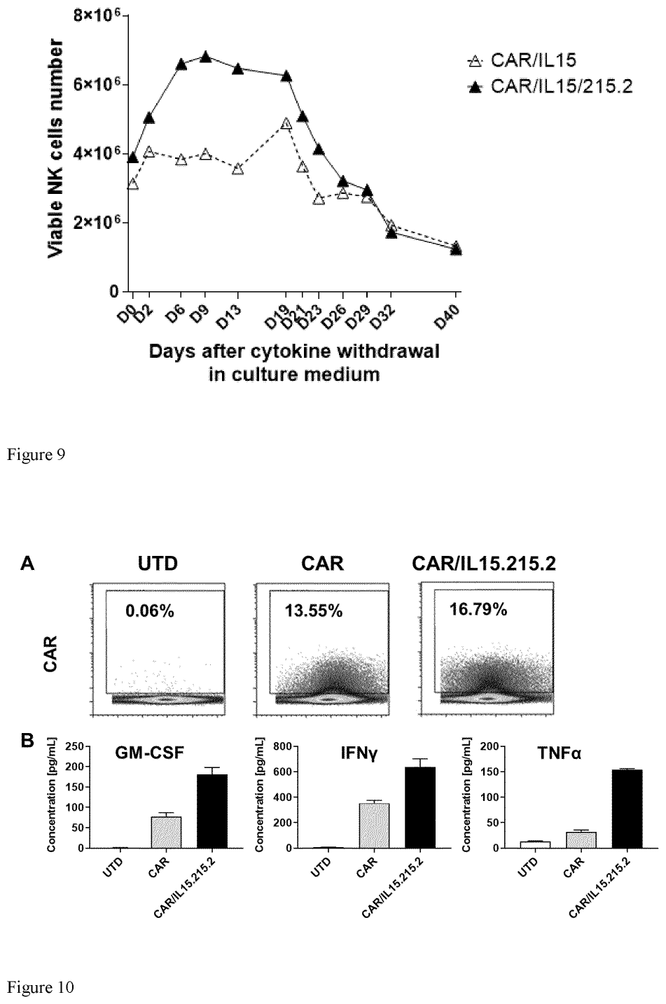

To assess whether the ectopic expression of IL15/215.2 gene may lead to dysregulated growth of gene-modified NK cells, CAR/IL15 and CAR/IL15/215.2 NK cells were cultured in NK cell culture medium without exogenous cytokine support. Both CAR/IL15 and CAR/IL15/215.2 NK cells expanded and maintained survival until day 19 in culture after cytokine withdrawal in the culture medium; however, the NK cell numbers continuously declined afterwards (FIG. 9).

The data summarized in Example 9 demonstrate that NK cells expressing IL15/215.2 did not induce autonomous or dysregulated growth, indicating the potential safety of this construct in the clinic.

### Example 10: Ectopic Expression of IL15.215.2 is Functional in Gene-Modified NK Cells

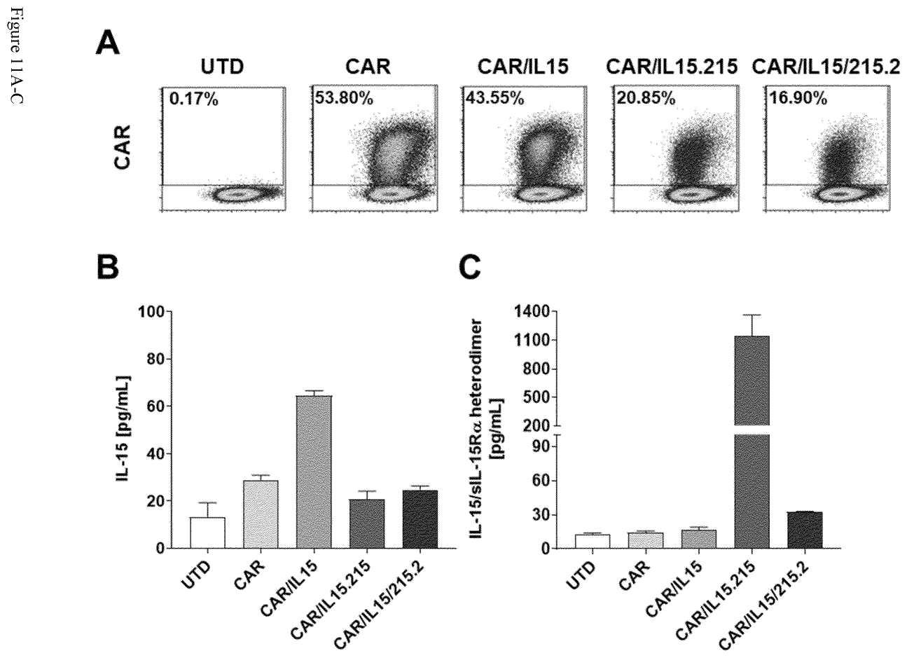

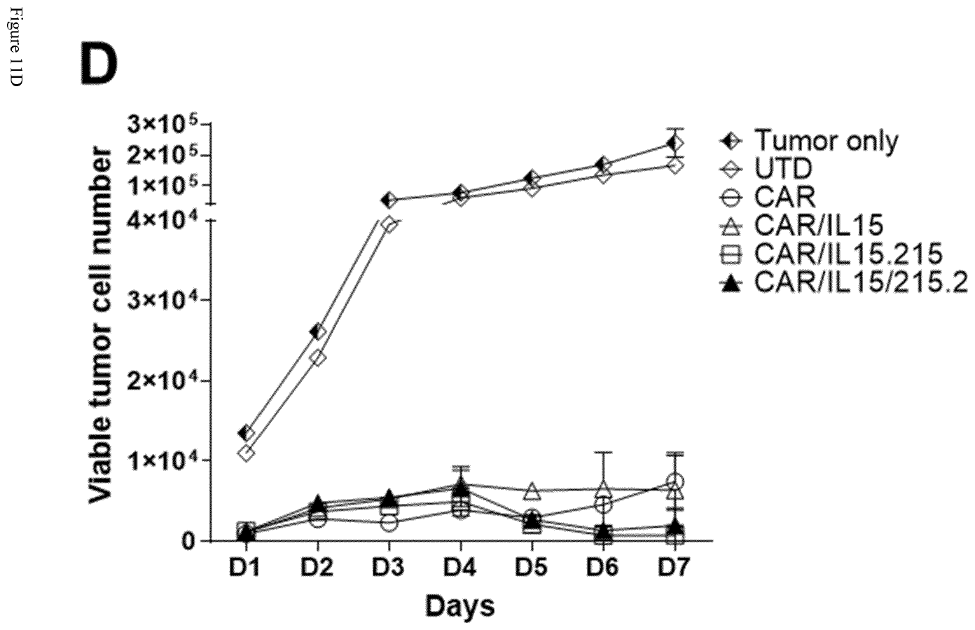

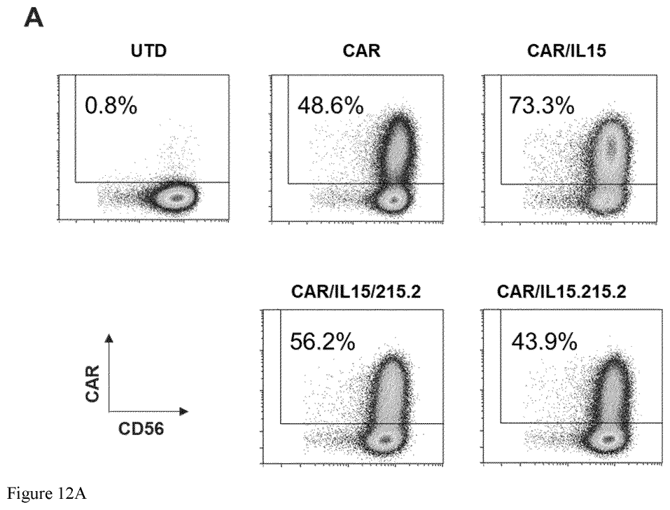

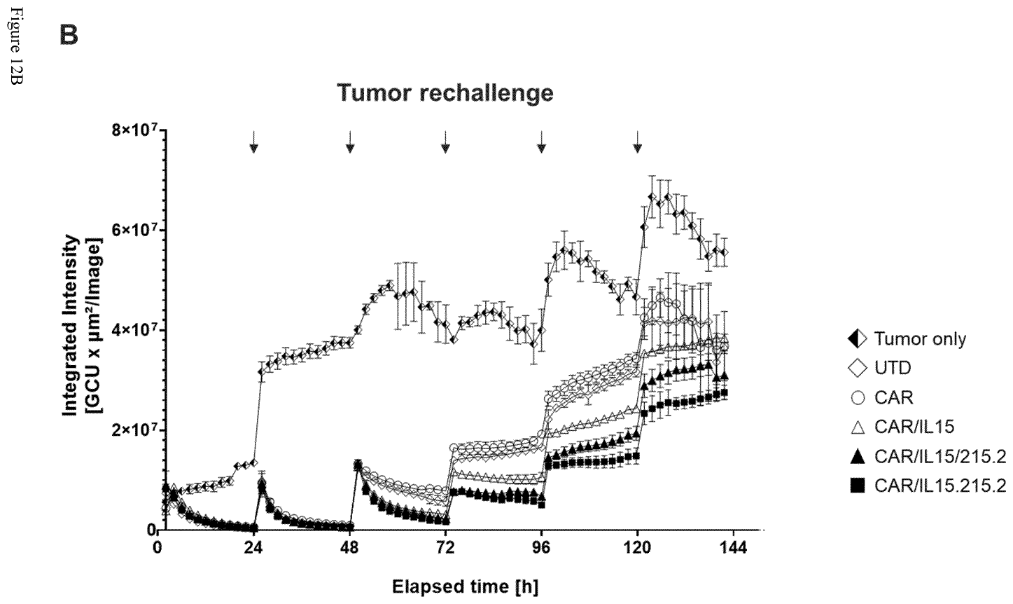

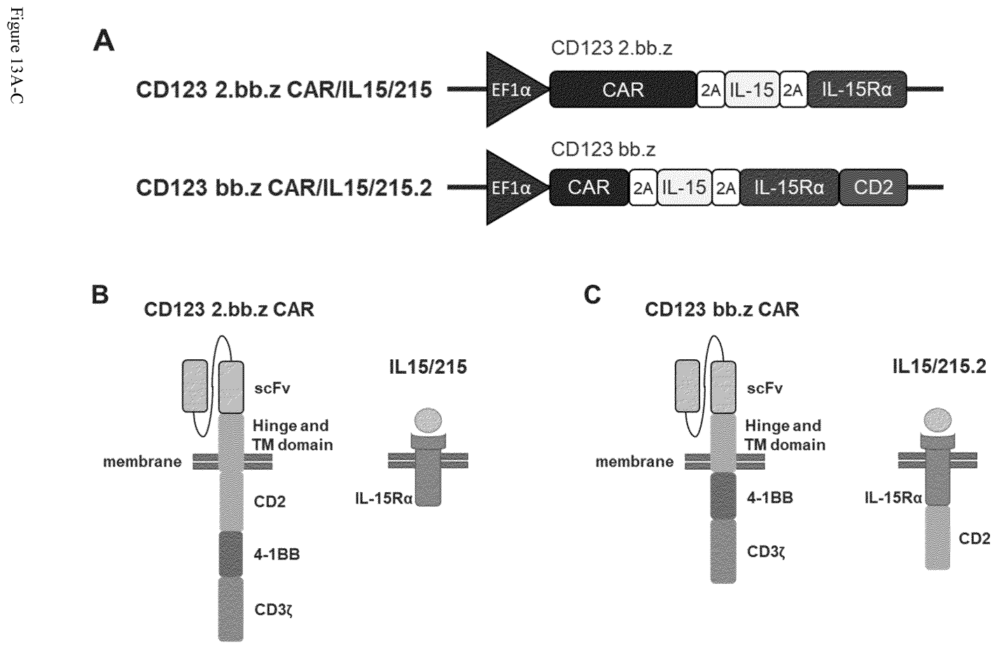

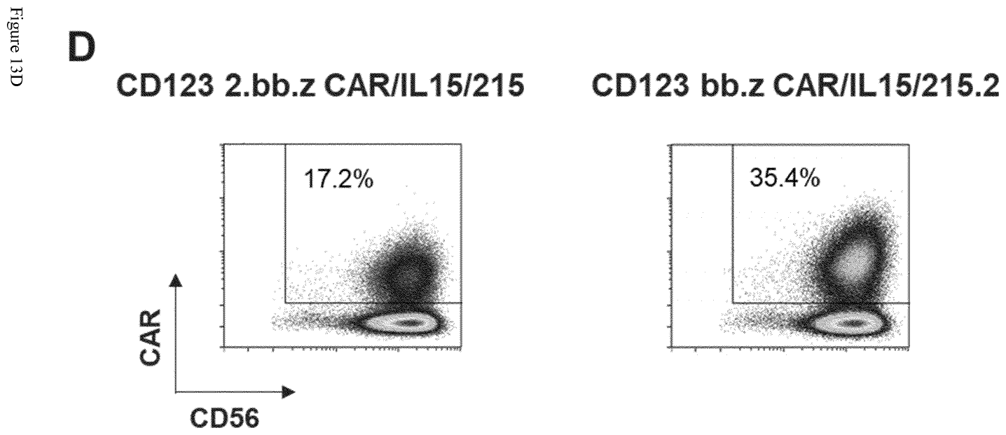

To investigate the functionality of IL15.215.2 construct, gene-modified NK cells were generated to express BDCA2 CAR construct (CAR NK) or coexpress BDCA2 CAR and IL15.215.2 constructs (CAR/IL15.215.2 NK) as described in Example 2. The CAR construct and IL15.215.2 construct have been explicated in Example 1 (FIG. 1). The surface CAR expressions on NK cells were determined at day 14 after isolation with flow cytometric analysis (FIG. 10A). CAR expression was detected by using biotinylated human recombinant BDCA2-Fc protein followed by VioBright515-conjugated anti-biotin antibody (Miltenyi Biotec). The percentage of CAR-positive NK cells in all the samples were adjusted by adding unmodified NK cells, according to the sample with the lowest transduction efficiency.

The functionality of CAR/IL15.215.2 NK cells was determined by the proinflammatory cytokine production of these cells. CAR-expressing NK cells were coincubated for 24 h with RS4;11/GFP/BDCA2 tumor cells at an E:T ratio of 1:1, which was calculated based on the number of transduced NK cells. UTD NK cells were included as control. Secreted cytokines in the culture supernatants were measured by using a MACSPlex Cytokine 12 Kit, human (Miltenyi Biotec), according to the manufacturer's instructions. The secretion of GM-CSF, IFNγ and TNFα were markedly upregulated for CAR-expressing NK cells, compared with UTD NK cells (FIG. 10B). Of note, CAR/IL15.215.2 NK cells produced higher cytokine levels, compared with NK cells only transduced with the CAR construct.

The data summarized in Example 10 demonstrate that IL15.215.2 is functional in gene-modified immune cells, indicating the potential use of this construct in the clinic.

### Example 11: Ectopic Expression of IL15/215.2 is Functional in Gene-Modified T Cells

To investigate the functionality of IL15/215.2 construct, gene-modified T cells were generated to express BDCA2 CAR construct or coexpress BDCA2 CAR and the complexes of IL-15:IL-15Rα variants. The CAR construct and the complexes of IL-15:IL-15Rα variants have been explicated in Example 1 (FIG. 1). In brief, T cells were purified from PBMCs by using the Pan T Cell Isolation Kit, human (Miltenyi Biotec) and activated in TexMACS™ medium (Miltenyi Biotec) containing T Cell TransAct™, human and 12.5 ng/ml of recombinant human IL-7 and 12.5 ng/ml of recombinant human IL-15 (all Miltenyi Biotec). Twenty-four hours after activation, T cells were transduced with vesicular stomatitis virus glycoprotein G (VSV-G) pseudotyped LVs-containing supernatants produced by transfected HEK293T cells. T cells were washed 2 days after transduction, transduced T cells were further cultured in TexMACS™ medium supplemented with 12.5 ng/mL of recombinant human TL-7 and 12.5 ng/ml of recombinant human IL-15 before use.

The surface CAR expressions on T cells were determined at day 14 after isolation with flow 20 cytometric analysis (FIG. 11A). CAR expression was detected by using biotinylated human recombinant BDCA2-Fc protein followed by VioBright515-conjugated anti-biotin antibody (Miltenyi Biotec).

Next, we assessed the secretion of soluble IL-15 and IL-15/sIL-15Rα heterodimers by gene-modified T cells. After 14 days of culture, CAR-expressing T cells were counted by flow cytometry and washed for 3 times by centrifugation with TexMACS medium. The percentage of CAR-positive T cells in all the samples were adjusted by adding unmodified T cells, according to the sample with the lowest transduction efficiency. The same number of CAR-positive T cells were seeded for all the samples in TexMACS medium without exogenous cytokine support and further cultivated for 24 hours. Supernatants were then collected and soluble IL-15 and IL-15/sIL-15Rα heterodimers were determined by using ELISA MAX™ Deluxe Set Human IL-15 kit (Biolegend) and Human IL-15/IL-15R alpha Complex DuoSet ELISA kit (R&D Systems), respectively, according to the manufacturers' instructions. IL-15 could be detected for all the T cell samples, which is in consistence with a previous study (Park et al. 2018, Immune Netw. 18, e13); however, CAR/IL15 T cells secreted the highest amount soluble IL-15 into the culture supernatants (FIG. 11B). The production of IL-15/sIL-15Rα heterodimers ranging from mean 13.09 to 16.4 pg/mL was detected for UTD, CAR and CAR/IL15 T cells, while a slightly elevated level of mean 32.47 pg/mL was secreted by CAR/IL15/215.2 T cells (FIG. 11C). The highest level of IL-15/sIL-15Rα heterodimers was observed in the supernatants derived from CAR/IL15.215 T cells.

To assess the potential of IL15/215.2 on maintaining the functionality of gene-modified T cells in the absence of exogenous cytokine support, the generated gene-modified T cells were coincubated with 3×10⁴ RS4;11/GFP/BDCA2 tumor cells at an initial E:T ratio of 1:2, which was calculated based on the number of transduced T cells. The gene-modified T cells were then repeatedly challenged for 7 days with 3×10⁴ fresh RS4;11/GFP/BDCA2 tumor cells in every 24 h. Gene-modified T cells and tumor cells were coincubated in RPMI1640 medium supplemented with 10% fetal bovine serum (FBS) and 2 mM L-Glutamine in the absence of cytokines in favor of tumor cell growth. At the time indicated in FIG. 11D, dead cells were excluded by using 7-AAD (Miltenyi Biotec) and the numbers of remaining viable target tumor cells were counted by using a MACSQuant Analyzer 10 flow cytometer. Target cells incubated without T cells and with UTD T cells were prepared to serve as controls. Tumor cells rapidly grew in the samples coincubated without T cells or with UTD T cells, while CAR-expressing T cells inhibited the tumor growth, resulting in the most efficient long-term tumor controls observed for CAR/IL15.215 and CAR/IL15/215.2 T cells (FIG. 11D).

The data summarized in Example 11 demonstrate that the expression of IL15/215.2 in CAR-expressing T cells can sustain their cytotoxicity against tumor cells in the absence of exogenous cytokine support, thereby promoting their continued anti-tumor response.

### Example 12: NK Cells Expressing IL15/215.2 or IL15.215.2 Show Sustained Natural Cytotoxicity Against Malignant Cells

Next, we assessed the long-term natural cytotoxicity of NK cells expressing IL15/215.2 or IIL15.215.2, which were generated as described in Example 2. The surface expression of BDCA2 CAR on NK cells was determined at day 14 after isolation as described in Example 2 (FIG. 12A). The percentage of CAR-positive NK cells in all the samples were adjusted by adding unmodified NK cells, according to the sample with the lowest transduction efficiency. To analyze the natural cytotoxicity of the gene-modified NK cells, a repetitive tumor challenge assay was performed. The gene-modified NK cells were coincubated with 3×10⁴ GFP-expressing K562 (K562/GFP) tumor cells at an initial E:T ratio of 2:1, which was calculated based on the number of transduced NK cells. The gene-modified NK cells were then repeatedly challenged with 3×10⁴ fresh K562/GFP tumor cells every 24 h for 6 days. Gene-modified NK cells and tumor cells were coincubated in RPMI1640 medium supplemented with 10% fetal bovine serum (FBS) and 2 mM L-Glutamine in the absence of cytokines in favor of tumor cell growth. Phase contrast and green fluorescence images were acquired by using the IncuCyte S3 system (Sartorius, Goettingen, Germany) until the end of the experiment.

Tumor cells rapidly grew in the samples without NK cells. All the NK cells showed similar tumor control within the first 48 h of coincubation. After that, CAR NK cells expressing IL-15 (CAR/IL15), IL15/215.2 (CAR/IL15/215.2) or IL15.215.2 (CAR/IL15.215.2) exhibited superior anti-tumor activity compared with UTD NK cells and CAR NK cells without IL-15 expression. Moreover, the best tumor growth control over time was observed with CAR/IL15/215.2 and CAR/IL15.215.2 NK cells (FIG. 12B).

The data summarized in Example 12 demonstrate that the expression of IL15/215.2 and IL15.215.2 in NK cells can sustain their natural cytotoxicity against tumor cells in the absence of exogenous cytokine support, thereby promoting their continued anti-tumor response.

### Example 13: The Advantage of Fusing the Intracellular CD2 Domain to the C-Terminus of the IL-15Rα Receptor

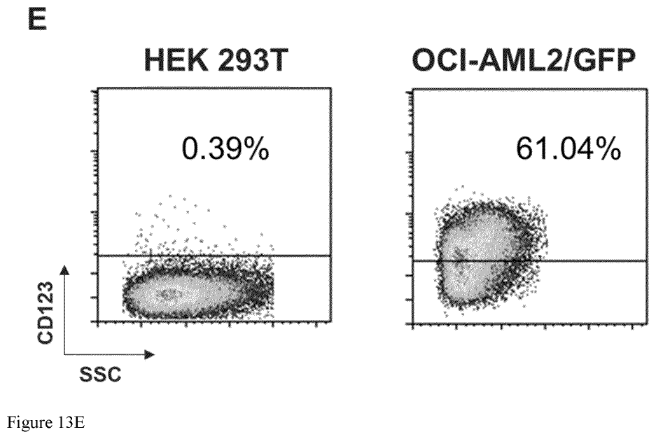

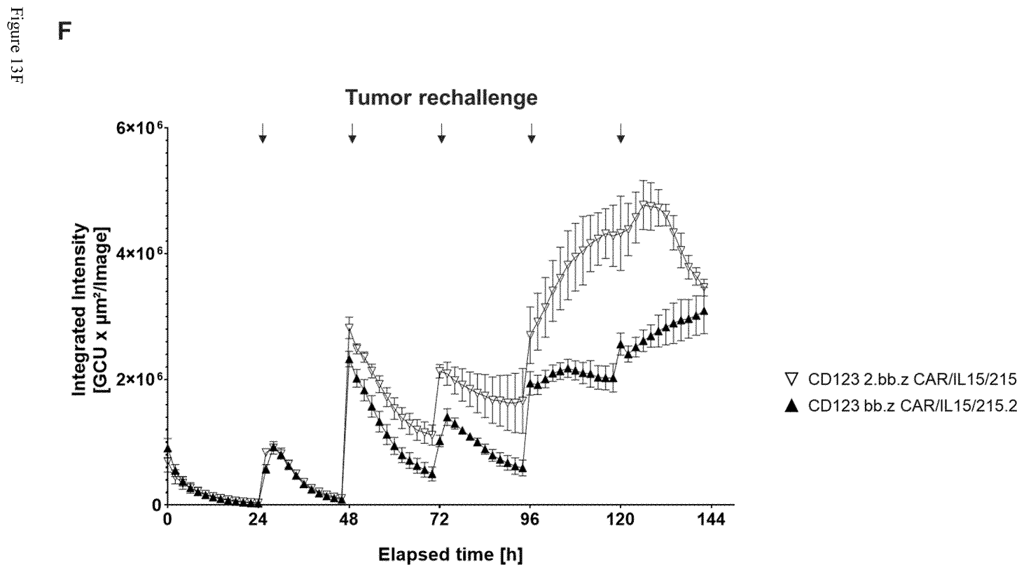

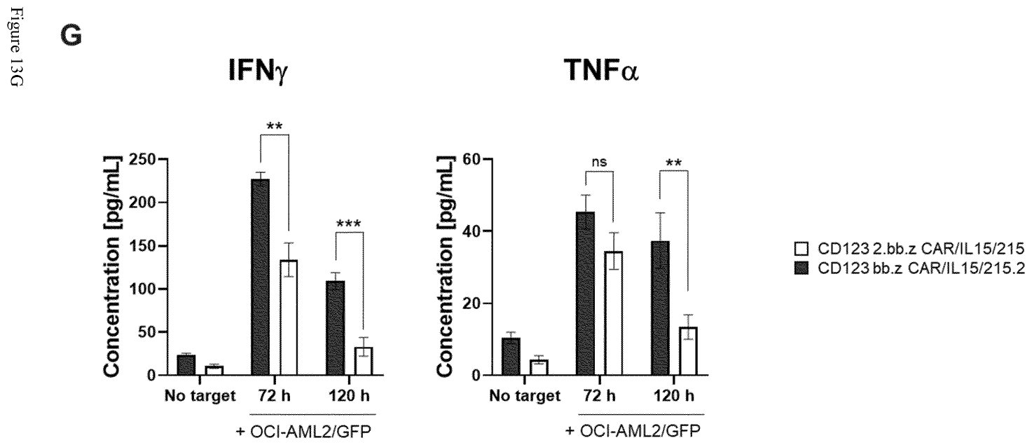

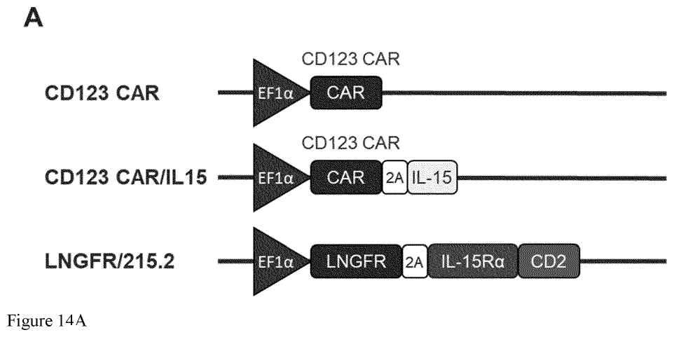

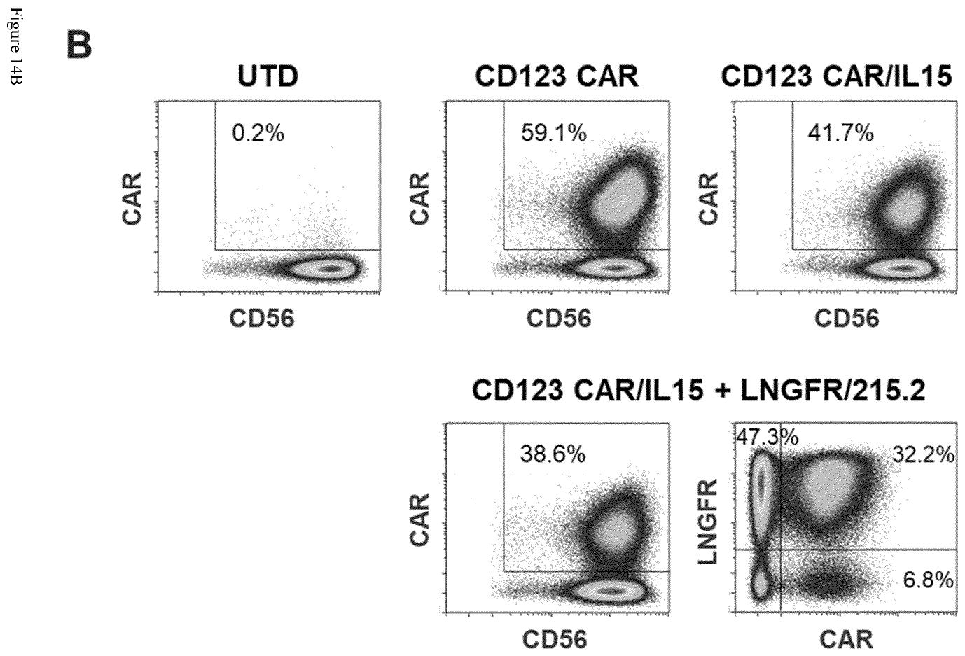

To assess whether the CD2 domain inserted into a CAR construct can provide a similar effect as when fused to the C-terminus of the IL-15Rα receptor, we compared NK cells transduced with the CD123 2.bb.z CAR/IL15/215 construct to NK cells transduced with the CD123 bb.z CAR/IL15/215.2 construct (FIG. 13A). The P2A system was used to separate CARs and IL-15, while the T2A system was used to separate IL-15 and IL-15Rα or IL-15Rα.CD2 as described in Example 1.

The CD123 2.bb.z CAR consists of a human granulocyte macrophage colony stimulating factor receptor alpha subunit (GM-CSFRa) signal peptide, a CD123-specific single chain Fv (scFv) antibody fragment, a CD8α hinge and transmembrane (TM) domain, followed by CD2, 4-1BB and CD3ζ intracellular (IC) domains (FIG. 13B), whereas the CD123 bb.z CAR is identical to the CD123 2.bb.z CAR except for the absence of the CD2 intracellular domain (FIG. 13C). NK cells were genetically engineered to co-express either CD123 2.bb.z CAR and IL15/215 or CD123 bb.z CAR and IL15/215.2 as described in Example 2. The surface expression of the CAR construct on NK cells was determined at day 14 after isolation by using human recombinant CD123-Fc protein, His tag (Acro Biosystems, Newark, DE, USA) followed by FITC-conjugated anti-His tag antibody (Miltenyi Biotec) (FIG. 13D). The percentage of CAR-positive NK cells in all the samples were adjusted by adding unmodified NK cells, according to the sample with the lowest transduction efficiency.

To analyze the cytotoxicity of the generated gene-modified NK cells, OCI-AML2 tumor cells engineered to express GFP (OCI-AML2/GFP) were used as target cells in a repetitive tumor challenge assay. The CD123 surface expression on OCI-AML2/GFP tumor cells was demonstrated by using APC-conjugated anti-CD123 antibody (REA918, Miltenyi Biotec). HEK293T cells served as control (FIG. 13E). The gene-modified NK cells were coincubated with 4×10⁴ OCI-AML2/GFP tumor cells at an initial E:T ratio of 2:1, which was calculated based on the number of transduced NK cells. The gene-modified NK cells were then repeatedly challenged with 4×10⁴ fresh OCI-AML2/GFP tumor cells every 24 h for 6 days. The experiment was performed in α-MEM Eagle with stable glutamine medium (PAN Biotech) supplemented with 20% fetal bovine serum (FBS) in the absence of cytokines in favor of tumor cell growth. The tumor growth was monitored by using the IncuCyte S3 system as described in Example 12. Both CD123 2.bb.z CAR/IL15/215 and CD123 bb.z CAR/IL15/215.2 NK cells showed similar tumor control within the first 48 h of coincubation. After that, CD123 bb.z CAR/IL15/215.2 NK cells showed superior anti-tumor activity compared with CD123 2.bb.z CAR/IL15/215 NK cells (FIG. 12F).

Supernatants were collected after 72 and 120 hours of coculture of CAR NK cells and OCI-AML2/GFP tumor cells and cytokine levels were measured using a MACSPlex Cytotoxic T/NK Cell Kit, human (Miltenyi Biotec) according to the manufacturer's instructions. NK cells without tumor cell stimulation were included as control. Compared with CD123 2.bb.z CAR/IL15/215 NK cells, CD123 bb.z CAR/IL15/215.2 NK cells secreted higher levels of IFNγ and TNFα upon tumor cell stimulation at both time points (FIG. 13G).

The data summarized in Example 13 demonstrate the advantage of fusing the intracellular CD2 domain to the C-terminus of the IL-15Rα receptor, compared with insertion into a CAR, indicating the critical role of the CD2 domain in the complexes of IL-15:IL-15Rα variants for optimal performance of gene-modified NK cells.

### Example 14: The IL-15:IL-15Rα.CD2 Complex is Functional when the IL-15 and IL-15Rα.CD2 Constructs are Encoded by Different Nucleic Acid Sequences

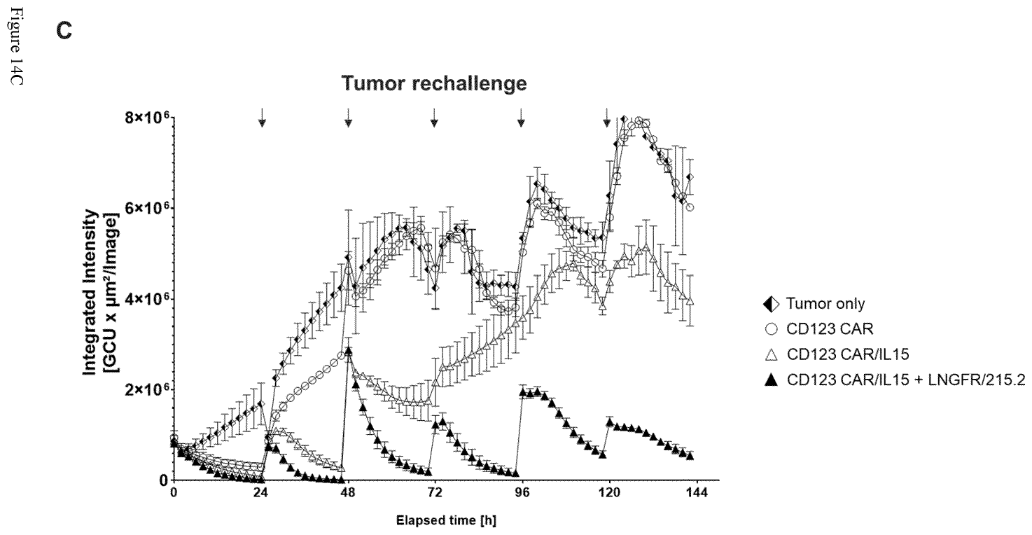

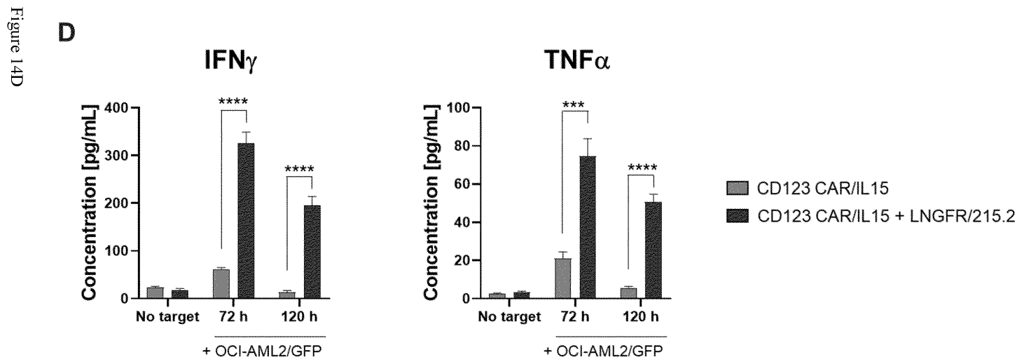

To assess the functionality of the IL-15:IL-15Rα.CD2 complex when the IL-15 and IL-15Rα.CD2 constructs are encoded by different nucleic acid sequences, we generated two DNA sequences, named as CD123 CAR/IL15 and LNGFR/215.2. The CD123 CAR/IL15 sequence consists of the CD123.bb.z CAR construct as described in Example 13 and native IL-15 as described in Example 1. CD123 CAR and IL-15 were separated by using a P2A self-cleaving peptide sequence. The LNGFR/215.2 sequence consists of a truncated low affinity nerve growth factor receptor (LNGFR) and the IL-15Rα.CD2 construct generated by fusing the CD2 intracellular domain to the C-terminus of IL-15Rα (215.2) as described in Example 1. A P2A sequence was used to separate LNGFR and 215.2. The 2A sequence and the native IL-15 sequence were deleted from the CD123 CAR/IL15 construct to generate the CD123 CAR sequence. The cassettes encoding CD123 CAR, CD123 CAR/IL15 and LNGFR/215.2 were then cloned into a self-inactivating, third generation lentiviral transfer plasmid backbone under the control of a human elongation factor 1 alpha (EF1α) promoter (FIG. 14A). BaEV pseudotyped LV-containing supernatants were produced by transient transfection of HEK 293T cells. LV-containing supernatants are stored at −80° C. before use.

CD123 CAR NK cells and CD123 CAR/IL15 NK cells were generated as described in Example 2. To generate NK cells co-expressing CD123 CAR/IL15 and LNGFR/215.2, purified primary NK cells were stimulated with NK cell culture medium supplemented with 2000 IU/mL of a human IL-1 family cytokine (Miltenyi Biotec) after isolation (Day 0). NK cells were then sequentially transduced with BaEV-LVs encoding LNGFR/215.2 and CD123 CAR/IL15 in the presence of Vectofusin®-1 at Day 1 and Day 2, respectively. The transduction efficiencies were determined by detecting the CAR expression as described in Example 13 and the LNGFR expression by using PE-Vio® 770-conjugated anti-LNGFR antibody (Miltenyi Biotec) (FIG. 14B). The percentage of CAR-positive NK cells in all the samples were adjusted by adding unmodified NK cells, according to the sample with the lowest CAR expression.

To analyze the cytotoxicity of the generated gene-modified NK cells, OCI-AML2/GFP tumor cells were used as target cells in a repetitive tumor challenge assay. The gene-modified NK cells were coincubated with 4×10⁴ OCI-AML2/GFP tumor cells at an initial E:T ratio of 2:1, which was calculated based on the number of CAR-expressing NK cells. The gene-modified NK cells were then repeatedly challenged with 4×10⁴ fresh OCI-AML2/GFP tumor cells every 24 h for 6 days. Tumor cells without any NK cells were prepared to serve as control. Gene-modified NK cells and tumor cells were coincubated in α-MEM Eagle with stable glutamine medium (PAN Biotech) supplemented with 20% fetal bovine serum (FBS) in the absence of cytokines in favor of tumor cell growth. The tumor growth was monitored by using the IncuCyte S3 system as described in Example 12. Tumor cells rapidly grew in the samples without NK cells. CD123 CAR NK cells and CD123 CAR/IL15 NK cells initially inhibited tumor outgrowth until tumor cells started to grow out at day 1 and day 2, respectively. In contrast, NK cells co-expressing CD123 CAR/IL15 and LNGFR/215.2 (CD123 CAR+LNGFR/215.2) showed improved long-term anti-tumor activity, maintaining strong control of tumor growth until the end of the experiment (FIG. 14C).

Supernatants were collected after 72 and 120 hours of coculture of NK cells and OCI-AML2/GFP tumor cells and cytokine levels were measured using a MACSPlex Cytotoxic T/NK Cell Kit, human (Miltenyi Biotec), according to the manufacturer's instructions. NK cells without tumor cell stimulation were included as control. Compared with CD123 CAR/IL15 NK cells, NK cells sequentially transduced with the LNGFR/215.2 and CD123 CAR/IL15 constructs secreted significantly higher levels of IFNγ and TNFα upon tumor cell stimulation at both time points (FIG. 14D).

The data summarized in Example 14 demonstrate the functionality of the IL-15:IL-15Rα.CD2 complex when the IL-15 and IL-15Rα.CD2 constructs are encoded by different nucleic acid sequences, highlighting the flexibility in the production of such gene-modified NK cells expressing the IL-15:IL-15Rα.CD2 complex.

### Example 15: No Autonomous or Dysregulated Growth was Observed in Gene-Modified NK Cells Co-Expressing the IL-15 and IL-15Rα.CD2 Constructs, which are Encoded by Different Nucleic Acid Sequences

To assess whether gene-modified NK cells co-expressing the IL-15 and IL-15Rα.CD2 constructs, which are encoded by different nucleic acid sequences, may exhibit dysregulated growth, the gene-modified NK cells generated in Example 14 were cultured in NK cell culture medium without exogenous cytokine support.

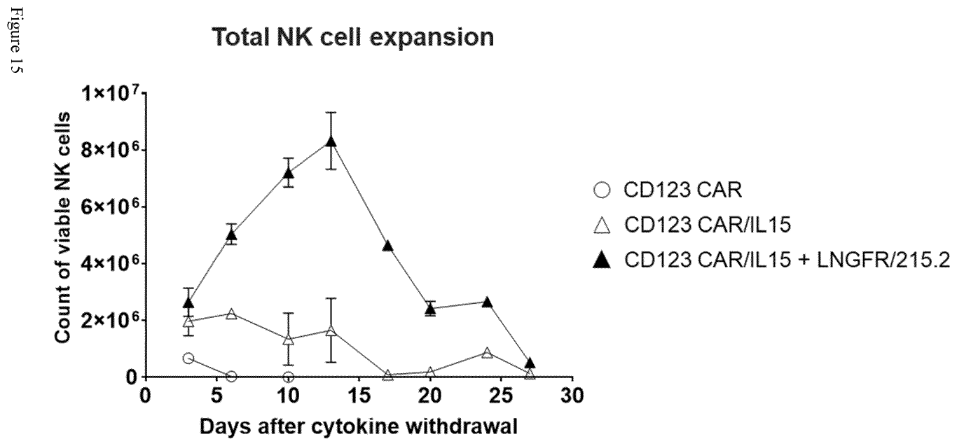

The gene-modified NK cells sequentially transduced with the LNGFR/215.2 and CD123 CAR/IL15 constructs (CD123 CAR/IL15+LNGFR/215.2) expanded and maintained survival until day 13 in culture after cytokine withdrawal, however, NK cell numbers continuously declined afterwards. In contrast, CD123 CAR NK cells showed much lower expansion potential in the absence of exogenous cytokine and CD123 CAR NK cells did not expand in the experiment (FIG. 15).

The data summarized in Example 15 demonstrate that co-expression of the IL-15 and IL15Rα.CD2 constructs by different nucleic acid sequences did not induce autonomous or dysregulated growth in gene-modified NK cells, indicating the potential safety of this application.

1-10. (canceled)

11. A composition that comprises an encoding region for a fusion protein and an encoding region for interleukin 15 (IL-15) in the same or in separate nucleic acid sequences,
wherein said fusion protein comprises in the direction from N to C terminus: interleukin-15 receptor subunit alpha (IL-15Rα), followed by a polypeptide that includes the intracellular signaling domain of CD2.

12. The composition according to claim 11, comprising separate encoding regions for the fusion protein and for the IL-15 in the same nucleic acid sequence.

13. The composition according to claim 11, wherein the IL-15 is part of the fusion protein.

14. The composition according to claim 13, wherein the fusion protein comprises in the direction from N to C terminus: (i) IL-15, (ii) a polypeptide linker, (iii) IL-15Rα, and (iv) a polypeptide that includes the intracellular signaling domain of CD2.

15. The composition according to claim 11, wherein the fusion protein and the IL-15 are encoded in separate nucleic acid sequences.

16. The composition according to claim 11, which further comprises a transgene.

17. The composition according to claim 16, wherein said transgene encodes a chimeric antigen receptor (CAR).

18. The composition according to claim 17, wherein the CAR is part of said fusion protein which also comprises the IL-15.

19. An immune cell that comprises an encoding region for a fusion protein and an encoding region for interleukin 15 (IL-15) in the same or in separate nucleic acid sequences,
wherein said fusion protein comprises in the direction from N to C terminus: interleukin-15 receptor subunit alpha (IL-15Rα), followed by a polypeptide that includes the intracellular signaling domain of CD2.

20. The immune cell according to claim 19, comprising separate encoding regions for the fusion protein and for the IL-15 in the same nucleic acid sequence.

21. The immune cell according to claim 19, wherein the IL-15 is part of the fusion protein.

22. The immune cell according to claim 21, wherein the fusion protein comprises in the direction from N to C terminus: (i) IL-15, (ii) a polypeptide linker, (iii) IL-15Rα, and (iv) a polypeptide that includes the intracellular signaling domain of CD2.

23. The immune cell according to claim 19, wherein the fusion protein and the IL-15 are encoded in separate nucleic acid sequences.

24. The immune cell according to claim 19, which further comprises a transgene.

25. The immune cell according to claim 24 wherein said transgene encodes a chimeric antigen receptor (CAR).

26. The immune cell according to claim 25, wherein the CAR is part of said fusion protein which also comprises the IL-15.

27. The immune cell according to claim 19, wherein said immune cell is an NK cell or a T cell.

28. A composition configured for administration to a human subject, comprising a plurality of immune cells according to claim 25 in combination with one or more pharmaceutically acceptable excipients.

29. The composition of claim 28, wherein the CAR is specific for a tumor associated antigen expressed on a cancer cell in the subject.

30. An in vitro method for preparing the composition of claim 28, comprising expanding a plurality of said immune cells in a culture medium without exogenous cytokine support.


SEQUENCE LISTING (12 sequences)

SEQ ID NO: 1 - protein, 29 aa, synthetic construct
MRISKPHLRSISIQCYLCLLLNSHFLTEA

SEQ ID NO: 2 - protein, 17 aa, synthetic construct
MPLLLLLPLLWAGALAM

SEQ ID NO: 3 - protein, 20 aa, synthetic construct
MYRMQLLSCIALSLALVTNS

SEQ ID NO: 4 - protein, 19 aa, synthetic construct
GIHVFILGCFSAGLPKTEA

SEQ ID NO: 5 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 7 - protein, 267 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 8 - protein, 237 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 9 - protein, 26 aa, synthetic construct
SGGGSGGGGSGGGGSGGGGSGGGSLQ

SEQ ID NO: 10 - protein, 116 aa, synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 11 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - protein, 131 aa, synthetic construct
MPLLLLLPLLWAGALAMNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS